(19) United States
(12) Patent Application Publication
(10) Pub. No.: US 2011/0252490 A1
(43) Pub. Date: Oct. 13, 2011

(54) Methods and Compositions for Increased Alpha-Prime Beta-Conglycinin Soybeans

(75) Inventor: Jonathan Jenkinson, Winnipeg (CA)

(73) Assignee: MONSANTO TECHNOLOGY LLC, St. Louis, MO (US)

(21) Appl. No.: 13/062,238

(22) PCT Filed: Sep. 1, 2009
(86) PCT No.: PCT/US09/55567

§ 371 Date: Jun. 23, 2011

Related U.S. Application Data
(60) Provisional application No. 61/094,277, filed on Sep. 4, 2008.

(51) Int. Cl. A01H 5/10; C40B 30/04; A01H 1/04

(52) U.S. Cl. 800/260; 800/312; 506/9

(57) ABSTRACT

The invention concerns methods for breeding soybean plants containing genomic regions associated with increased α′-subunit of β-conglycinin content in seed. Moreover, the invention provides germplasm and the use of germplasm containing genomic regions conferring increased α′-subunit of β-conglycinin content for introgression into elite germplasm in a breeding program. The invention also provides derivatives, and plant parts of these plants and uses thereof.

### CROSS-REFERENCE TO RELATED APPLICATIONS

This application claims the benefit under 35 U.S.C. §119(e) of U.S. Provisional Application No. 61/094,277 filed Sep. 4, 2008. The entirety of the application is hereby incorporated by reference.

### BACKGROUND OF THE INVENTION

1. Incorporation of the Sequence Listing

A sequence listing is contained in the file named “pa_{—}53703D.txt'” which is 38,062 bytes (measured in MS-Windows) and was created on Aug. 22, 2009. This electronic sequence listing is electronically filed herewith and is incorporated herein by reference.

2. Field of the Invention The present invention relates generally to the field of plant breeding and molecular biology. In particular, the invention relates to soybeans with increased α′ subunit of β-conglycinin content and materials for making such plants. More specifically, the invention includes a method for breeding soybean plants containing quantitative trait loci that are associated with increased α′ subunit. The invention further includes germplasm and the use of germplasm containing quantitative trait loci (QTL) conferring increased α′ subunit for introgression into elite germplasm in a breeding program.

### 3. DESCRIPTION OF RELATED ART

US growers plant two types of soybeans, oil/meal and food grade. The oil/meal beans are grown primarily for the U.S. market. The soy used as a food ingredient is typically in the form of flour, concentrate, isolate, or oil. The soy ingredients are highly sought after because of their functionality, nutritional properties, low cost, and abundance (Zayas et al., Functionality of Proteins in Food, (1997).

Composition and conformation are responsible for a protein's functionality. Compositional differences that could alter functionality include, for example, the ratio of protein fractions, variations in subunit concentrations within fractions, and differences in amino acid profiles. Soy proteins have four major water-extractable fractions (2S, 7S, 11S, and 15S) that can be isolated on the basis of their sedimentation coefficients. The 7S (β-conglycinin) and 11S (glycinin) proteins represent the majority of the fractions within the soybean.

The glycinin (11s globulin) is composed of five different subunits, designated A1aB2. A2B1a, A1bB1b, A5A4B3, A3B4, respectively. Each subunit is composed of two polypeptides, one acidic and one basic, covalently linked through a dulfide bond. The two polypeptide chains result from post-translational cleavage of proglycinin precursors; a step that occurs after the precursor enters the protein bodies. Five major genes have been identified to encode these polypeptide subunits. They are designated as Gy1, Gy2, Gy3, Gy4 and Gy5, respectively (Nielsen et al., In: Cellular and molecular biology of plant seed development, Larkins and Vasil 1K (Eds)., Kluwer Academic Publishers, Dordrecht, The Netherlands, 151-220 (1997). In addition, a pseudogene, gy6, and minor gene, Gy7, were also reported (Beilinson et al., Theor. Appl. Genet., 104 (6-7):1132-1140 (2002). Genetic mapping of these genes has been reported by various groups (Diers et al. 1993, Chen and Shoemaker 1998, Beilinson et al, 2002). Gy1 and Gy2 were located 3 kb apart and mapped to linkage group N (Nielsen et al., Plant Cell., 1:313-328 (1989) Gy3 was mapped to linkage group L Beilinson et al., Theor. Appl. Genet, 104 (6-7):1132-1140 (2002). Gy4 and Gy5 were mapped to linkage groups O and F, respectively.

β-conglycinin (7S), on the other hand, is composed of α (˜67 kda), α′ (˜71 kDa) and β (˜50 kDa) subunits and each subunit is processed by co- and post-translational modifications (Ladin et al., Plant Physil., 84:35-41 (1987); Utsumi, In: Advances in Food and Nutrition Research, Kinsella (Ed.). 36:89-208, Academic Press, San Diego, Calif. (1992). The β-conglycinin subunits are encoded by the genes Cgy1, Cgy2 and Cgy3, respectively. Genetic analysis indicated that Cgy2 is tightly linked to Cgy3, whereas Cgy1 segregates independently of the other two. Cgy1 is associated with the α′-subunit (Doyle et al., J Biol Chem: 261: 9225-9238 (1986); and Cgy3 is associated with the α-subunit (Yoshino et al., Genes Genet. Syst. 76: 99-105 (2001). In addition, the down regulation Cgy3 results in the upregulation of Cgy1, Hew, a mutation in Cgy3 resulting in reduce α-subunit accumulation, may result incread α′-subunit accumulation. The β-conglycinin gene family contains at least 15 members divided into two major groups, which encode the 2.5-kb and 1.7-kb embryo mRNA, respectively (Harada et al., Japan J. Breed., 33:23 30 (1983). The relative percentages of α′, α, and β chains in the trimer are ˜35, 45, and 20% of total β-conglycinin, respectively (Maruyama et al., J. Agric. Food Chem. 47:5278-528 (1999).

Soy protein functionality is partly dependent on the β-conglycinin-to-glycinin ratio and variations in the subunit compositions, which can vary among genotypes. The differences in composition and structure between β-conglycinin and glycinin are exhibited in both nutritional and functional properties. Glycinins contains more methionine and cysteine per unit than β-conglycinins, however soybeans lacking glycinins and enriched in beta-conglycinins can have similar levels of total sulfur amino acids as soybeans containing glycinins Glycinins are important for forming the protein particles that make up firm tofu gels (Tezuka et al., J. Agric. Food Chem., 48:1111-1117 (2000), but weaker gels are formed in the absence of beta-conglycinin than those formed in the absence of glycinins (Tezuka et al., J. Agric. Food Chem., 52:1693-1699 (2004). The gelling properties of β-conglycinins and of soy protein isolates made from soybeans enriched in β-conglycinins show advantages under some conditions that may apply to meat applications (Nagano, et al., J. Agric. Food Chem. 44:3484-3488 (1996); Rickert, et al. J. Fd Sci. 69:303 (2004). The gelling properties of β-conglycinin can be altered by varying the subunit composition with the alpha-subunit showing advantages (Salleh, 2004). The solubility and emulsifying properties of β-conglycinin are good in part because of the hydrophilic extention regions of α and α′ subunits (Yamauchi et al., Food Rev. Int. 7: 283-322 (1991), Maruyama et al., JAOCS. 79:139 (2002). There is potential to create valuable soybeans and ingredients for food use having increased β-conglycinin levels and decreased glycinin levels.

β-conglycinin has significant potential to positively impact human health (Baba et al., J. Nutr. Sci. Vitaminol. (Tokyo), 50(1):26-31 (2004). In particular, β-conglycinin has been found to lower cholesterol, triglycerides and visceral fat. Kohno et al. demonstrated that a significant reduction in triglycerol levels and viseral fat in human subjects that consumed 5 g of β-conglycinin per day (Kohno et al., J Atheroscler Thromb, 13: 247-255 (2006). Similarly, Nakamura et al. found that β-conglycinin up-regulates genes associated with lipid metabolism in a primate model (Nakamura et al., Soy Protein Res 8: 1-7 (2005). In addition, Nakamura et al. showed β-conglycinin had a significant effect preventing bone mineral density loss (Nakamura et al., Soy Protein Res 7: 13-19 (2004). In addition, β-conglycinin demonstrated effects in lowering serum insulin and blood sugar (Moriyama et al., Biosci. Biotechnol. Biochem., 68(2):352-359 (2004). Due to β-conglycinin effects on triglycerides, cholesterol, fat, insulin and sugar levels, it may play an important role in health programs. In addition, β-conglycinin inhibits artery plaque formation in mice and may have similar affects in human subjects as well (Adams et al., J. Nutr., 134(3):511-516 (2004). Furthermore, β-conglycinin may have a significant effect on intestinal microflora in humans. β-conglycinin is inhibits growth of harmful bacteria, such as E. coli while stimulating growth of beneficial bacteria, such as bifidobacteria, in a number of animal models (Nakamura et al., Soy Protein Res 7: 13-19 (2004), Zuo et al., World J Gastroenterol 11: 5801-5806 (2005). β-conglycinin could be used both to reduce E. coli growth after infection and maintain a healthy intestinal microbial community.

The α′ subunit of β-conglycinin may play a predominant role in many of the health benefits associated with β-conglycinin. A number of experiments using animal models have indicated that α′ subunit from soybean β-conglycinin could lower plasma triglycerides, and also increase LDL (“bad” cholesterol) removal from blood (Duranti et al., J. Nutr. 134(6):1334-1339 (2004), Moriyama et al. Biosci. Biotechnol Biochem., 68(2):352-359 (2004), Adams et al., J. Nutr., 134(3):511-516 (2004), Nishi et al., J. Nutr., 133(2):352-357 (2003). Therefore, soybean varieties with an increased β-conglycinin content will have higher value than traditional varieties and will be suitable for use in nutrition drinks and other food products. In an attempt to identify the biologically active polypeptide(s), Manzoni et al. attempted to characterize biologically active polypeptides in β-conglycinin and indirectly demonstrated that the α′-subunit had a putative role in lowering cholesterol (Manzoni et al., J. Agric. Food Chem 46:2481-2484 (1998). Additionally, Manzoni also demonstrated the influence of the α′ subunit on the increase in LDL uptake and degradation and LDL receptor mRNA levels (Manzoni et al. J. Nutr. 133:2149-2155 (2003). Duranti et al. demonstrated that the α′ subunit can lower triglycerides and plasma cholesterol in vivo (Duranti et al., J. Nutr. 134(6):1334-1339 (2004).

The β-subunit of β-conglycinin has a number of health benefits as well. For instance, the β-subunit enhances satiety by causing cholecystokinin secretion (Nishi et al. J. Nun. 133:352-357 2003, Hara et al. Plant Phys Biochem 42: 657-662 (2004). Cholecystokinin is a peptide hormone of the gastrointestinal system responsible for stimulating the digestion of fat and protein. Cholecystokinin, previously called pancreozymin, is synthesized by 1-cells and secreted in the duodenum, the first segment of the small intestine, and causes the release of digestive enzymes and bile from the pancreas and gallbladder, respectively. It also acts as a hunger suppressant. Hence. β-subunit may suppress appetite and may play a role in an overall weight management program.

The β-subunit may have a function in mental health as well. Soymorphin-5 are released by digesting the β-subunit with pancreatic elastase and leucine aminopeptidase. Soymorphin-5 is an opioid peptide. Opioids are chemical substances that have a morphine-like action in the body. Opioids are primarily used for pain relief. These agents work by binding to opioid receptors, which are found principally in the central nervous system and the gastrointestinal tract. Soymorphin-5 demonstrated anxiolytic effect after oral administration on mice, which suggest the intake of β-subunit may decrease mental stress (Agui et al. Peptide Science 2005: 195-198 (2005).

Thus, it is an objective of the present invention to produce soybeans with increased levels of the α′-subunit of β-conglycinin The present invention provides and includes a method for screening and selecting a soybean plant comprising QTL for altered levels of α′-subunit and single nucleotide polymorphisms (SNP) marker technology.

### SUMMARY OF THE INVENTION

The present invention relates to increased α′-subunit and conserved 3 subunit composition of soybean seed which has improved physical and human health properties compared commercial soybean protein ingredients. The current invention provides methods for selecting a soybean plant with non-transgenic traits conferring increased α′-subunit phenotype and decreased seed α-subunit content. Thus, the methods of the current invention comprise, in one aspect, selecting seeds with increased α′-subunit content and decreased α-subunit content. In certain embodiments, the seed α′-subunit content for plants of the invention is about or at least about 9, 10, 11, 12, 13, 14, 15, 16, 17, 18, 19, 20 percent or more of the total protein content. In some embodiments, a plant of the invention has a ratio of α-subunit content to α′-subunit of about 1.0, 0.9, 0.8, 0.7, 0.6, 0.5, 0.4, 0.3, 0.2, 0.1 or even 0, derivable therein.

The present invention includes methods for introgressing alleles into a soybean plant comprising (a) crossing at least a first soybean plant comprising a nucleic acid sequence selected from those listed in Table 3 with at least a second soybean plant in order to form a segregating population, (b) screening the segregating population with one or more nucleic acid markers to determine if one or more soybean plants from the segregating population contains a listed nucleic acid sequence, and (c) selecting from that segregating population one or more soybean plants comprising a nucleic acid sequence selected from those that are listed in Table 3.

The present invention includes methods for introgressing alleles and selecting for non-transgenic traits conferring increased α′-subunit phenotype in seed of a soybean plant comprising (a) crossing at least one soybean plant with increased seed α′-subunit content in seed with a second soybean plant in order to form a segregating population and (b) screening the segregating population with one or more nucleic acid markers to determine if one or more soybean plants from the segregating population contain alleles of genomic region associated with increased α′-subunit phenotype and increased seed α′-subunit content in seed.

The present invention further provides a method for selection and introgression of genomic regions associated with a non-transgenic traits conferring decreased α-subunit content resulting in increased α′-subunit phenotype in seed of comprising: (a) isolating nucleic acids from a plurality of soybean plants; (b) detecting in the isolated nucleic acids the presence of one or more marker molecules wherein the marker molecule is selected from the group consisting of SEQ ID NO: 1 through SEQ ID NO: 18, and any one maker molecule mapped within 30 cM or less from the marker molecules; and (c) selecting a soybean plant comprising the one or more marker molecules, thereby selecting a soybean plant of with increased seed α′-subunit content in seed.

The current invention provides, as a further embodiment, methods for selecting soybean plants capable of producing seeds with reduced glycinin content, increased seed β-conglycinin content and subsequently increased α′-subunit of β-conglycinin. Thus, the plants of the current invention comprise, in one aspect, seeds with reduced glycinin content, increased β-conglycinin content and α′-subunit of β-conglycinin. In some embodiments, a plant of the invention produces a seed comprising a seed glycinin content of about or less than about 18, 17, 16, 15, 14, 13, 12, 11, 10, 9, 8, 7, 6, 5, 4, 3, 2, 1, or 0 percent of the total seed protein. In certain embodiments, the plant of the current invention produces a seed comprising a seed β-conglycinin content of about or at least about 37, 38, 39, 40, 41, 42, 43, 44, 45, 46, 47, 48, 49, 50, 51, 52, 53, 54, 55, 56, 57, 58, 59, or 60 percent or more of the total seed protein. In another embodiment, the seed α′-subunit content for plants of the invention is about or at least about 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 percent or more of the total seed protein content. In further embodiments, a plant of the invention is capable of producing a seed with a β-conglycinin content comprising an α-subunit and an α′-subunit in a ratio of about 1.0, 0.9, 0.8, 0.7, 0.6, 0.5, 0.4, 0.3, 0.2, 0.1 or even 0.

The present invention includes methods for introgressing alleles and selecting for with reduced glycinin content, increased seed β-conglycinin content and subsequently increased α′-subunit of β-conglycinin content in seed of a soybean plant comprising (a) crossing at least one soybean plant with reduced glycinin content, increased seed β-conglycinin content and subsequently increased α′-subunit of β-conglycinin content in seed with a second soybean plant in order to form a segregating population and (b) screening the segregating population with one or more nucleic acid markers to determine if one or more soybean plants from the segregating population contain alleles of genomic region associated with reduced glycinin content, increased seed β-conglycinin content and subsequently decreased α-subunit protein content resulting in increased α′-subunit of β-conglycinin content in seed.

The present invention further provides a method for selection and introgression of genomic regions associated with a with reduced glycinin content, increased seed β-conglycinin content and subsequently increased α′-subunit of β-conglycinin content in seed of comprising: (a) isolating nucleic acids from a plurality of soybean plants; (b) detecting in the isolated nucleic acids the presence of one or more marker molecules wherein the marker molecule is selected from the group consisting of SEQ ID NO: 1 through SEQ ID NO: 18, and any one maker molecule mapped within 30 cM or less from the marker molecules; and (c) selecting a soybean plant comprising the one or more marker molecules, thereby selecting a soybean plant of with reduced glycinin content, increased seed β-conglycinin content and subsequently decreased α-subunit protein content resulting in increased α′-subunit of β-conglycinin content in seed.

Plant parts are also provided by the invention. Parts of a plant of the invention include, but are not limited to, pollen, ovules, meristems, cells, and seed. Cells of the invention may further comprise, regenerable cells, such as embryos meristematic cells, pollen, leaves, roots, root tips, and flowers. Thus, these cells could be used to regenerate plants of the invention.

Also provided herein are parts of the seeds of a plant according to the invention. Thus, crushed seed, and meal or flour made from seed according to the invention is also provided as part of the invention. The invention further comprises, a method for making soy meal or flour comprising crushing or grinding seed according to the invention. Such soy flour or meal according to the invention may comprise genomic material of plants of the invention. In one embodiment, the food may be defined as comprising the genome of such a plant. In further embodiments soy meal or flour of the invention may be defined as comprising increased β-conglycinin and decreased glycinin content, as compared to meal or flour made form seeds of a plant with an identical genetic background, but not comprising the non-transgenic, mutant Gy3 and Gy4 null alleles.

In yet a further aspect of the invention there is provided a method for producing a soybean seed, comprising crossing the plant of the invention with itself or with a second soybean plant. Thus, this method may comprise preparing a hybrid soybean seed by crossing a plant of the invention with a second, distinct, soybean plant.

Still yet another aspect of the invention is a method of producing a food product for human or animal consumption comprising: (a) obtaining a plant of the invention; (b) cultivating the plant to maturity; and (c) preparing a food product from the plant. In certain embodiments of the invention, the food product may be protein concentrate, protein isolate, meal, flour or soybean hulls. In some embodiments, the food product may comprise beverages, infused foods, sauces, coffee creamers, cookies, emulsifying agents, bread, candy instant milk drinks, gravies, noodles, soynut butter, soy coffee, roasted soybeans, crackers, candies, soymilk, tofu, tempeh, baked soybeans, bakery ingredients, beverage powders, breakfast cereals, nutritional bars, meat or meat analogs, fruit juices, desserts, soft frozen products, confections or intermediate foods. Foods produced from the plants of the invention may comprise increased α′-subunit content and thus be of greater nutritional value foods made with typical soybean varieties

In a further aspect of the invention is a method of producing a nutraceutical, comprising: (a) obtaining a plant of the invention; (b) cultivating the plant to maturity; and (c) preparing a nutraceutical from the plant. Products produced from the plants of the invention may comprise increased α′-subunit content and thus be of greater nutritional value foods made with typical soybean varieties. For example, products from soybean seeds with increased α′-subunit may be used alone or combination with other mechanisms in a lipid-lowering therapy.

In further embodiments, a plant of the invention may further comprise a transgene. The transgene may in one embodiment be defined as conferring preferred property to the soybean plant selected from the group consisting of herbicide tolerance, increased yield, insect control, fungal disease resistance, virus resistance, nematode resistance, bacterial disease resistance, mycoplasma disease resistance, altered fatty acid composition, altered oil production, altered amino acid composition, altered protein production, increased protein production, altered carbohydrate production, germination and seedling growth control, enhanced animal and human nutrition, low raffinose, drought and/or environmental stress tolerance, altered morphological characteristics, increased digestibility, industrial enzymes, pharmaceutical proteins, peptides and small molecules, improved processing traits, improved flavor, nitrogen fixation, hybrid seed production, reduced allergenicity, biopolymers, biofuels, or any combination of these.

In certain embodiments, a plant of the invention may be defined as prepared by a method wherein a plant comprising non-transgenic mutations conferring increased α′-subunit phenotype and decreased α-subunit content is crossed with a plant comprising agronomically elite characteristics. The progeny of this cross may be assayed for agronomically elite characteristics and α′-subunit protein content, and progeny plants selected based on these characteristics, thereby generating the plant of the invention. Thus in certain embodiments, a plant of the invention may be produced by crossing a selected starting variety with a second soybean plant comprising agronomically elite characteristics. In some embodiments, a plant of the invention may be defined as prepared by a method wherein a plant comprising a non-transgenic mutation conferring a reduced glycinin content and an increased seed β-conglycinin content is crossed with a plant comprising increased α′-subunit content.

Embodiments discussed in the context of a method and/or composition of the invention may be employed with respect to any other method or composition described herein. Thus, an embodiment pertaining to one method or composition may be applied to other methods and compositions of the invention as well.

As used in the specification or claims, “a” or “an” may mean one or more. As used herein in the claim(s), when used in conjunction with the word “comprising”, the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1: Influence of markers associated with increased α′-subunit content on the alpha subunit content as measured by SDS-PAGE.

FIG. 2: Influence of markers associated with increased α′-subunit content on the ratio of α/α′-subunits as measured by SDS-PAGE.

### BRIEF DESCRIPTION OF NUCLEIC ACID SEQUENCES

SEQ ID NO: 1 is a genomic sequence derived from Glycine max (L.) Merrill corresponding to a genomic region associated with decreased α-subunit levels.

SEQ ID NO: 2 is a genomic sequence derived from Glycine max (L.) Merrill corresponding to a genomic region associated with decreased α-subunit levels.

SEQ ID NO: 3 is a genomic sequence derived from Glycine max (L.) Merrill corresponding to a genomic region associated with decreased α-subunit levels.

SEQ ID NO: 4 is a genomic sequence derived from Glycine max (L.) Merrill corresponding to a genomic region associated with decreased α-subunit levels.

SEQ ID NO: 5 is a genomic sequence derived from Glycine max (L.) Merrill corresponding to a genomic region associated with decreased α-subunit levels.

SEQ ID NO: 6 is a genomic sequence derived from Glycine max (L.) Merrill corresponding to a genomic region associated with decreased α-subunit levels.

SEQ ID NO: 7 is a genomic sequence derived from Glycine max (L.) Merrill corresponding to a genomic region associated with decreased α-subunit levels.

SEQ ID NO: 8 is a genomic sequence derived from Glycine max (L.) Merrill corresponding to a genomic region associated with decreased α-subunit levels.

SEQ ID NO: 9 is a genomic sequence derived from Glycine max (L.) Merrill corresponding to a genomic region associated with decreased α-subunit levels.

SEQ ID NO: 10 is a genomic sequence derived from Glycine max (L.) Merrill corresponding to a genomic region associated with decreased α-subunit levels.

SEQ ID NO: 11 is a genomic sequence derived from Glycine max (L.) Merrill corresponding to a genomic region associated with decreased α-subunit levels.

SEQ ID NO: 12 is a genomic sequence derived from Glycine max (L.) Merrill corresponding to a genomic region associated with decreased α-subunit levels.

SEQ ID NO: 13 is a genomic sequence derived from Glycine max (L.) Merrill corresponding to a genomic region associated with decreased α-subunit levels.

SEQ ID NO: 14 is a genomic sequence derived from Glycine max (L.) Merrill corresponding to a genomic region associated with decreased α-subunit levels.

SEQ ID NO: 15 is a genomic sequence derived from Glycine max (L.) Merrill corresponding to a genomic region associated with decreased α-subunit levels.

SEQ ID NO: 16 is a genomic sequence derived from Glycine max (L.) Merrill corresponding to a genomic region associated with decreased α-subunit levels.

SEQ ID NO: 17 is a genomic sequence derived from Glycine max (L.) Merrill corresponding to a genomic region associated with decreased α-subunit levels.

SEQ ID NO: 18 is a genomic sequence derived from Glycine max (L.) Merrill corresponding to a genomic region associated with decreased α-subunit levels.

SEQ ID NO: 19 is a PCR primer for amplifying SEQ ID NO: 1.

SEQ ID NO: 20 is a PCR primer for amplifying SEQ ID NO: 1.

SEQ ID NO: 21 is a PCR primer for amplifying SEQ ID NO: 2.

SEQ ID NO: 22 is a PCR primer for amplifying SEQ ID NO: 2.

SEQ ID NO: 23 is a PCR primer for amplifying SEQ ID NO: 3.

SEQ ID NO: 24 is a PCR primer for amplifying SEQ ID NO: 3.

SEQ ID NO: 25 is a PCR primer for amplifying SEQ ID NO: 4.

SEQ ID NO: 26 is a PCR primer for amplifying SEQ ID NO: 4.

SEQ ID NO: 27 is a PCR primer for amplifying SEQ ID NO: 5.

SEQ ID NO: 28 is a PCR primer for amplifying SEQ ID NO: 5.

SEQ ID NO: 29 is a PCR primer for amplifying SEQ ID NO: 6.

SEQ ID NO: 30 is a PCR primer for amplifying SEQ ID NO: 6.

SEQ ID NO: 31 is a PCR primer for amplifying SEQ ID NO: 7.

SEQ ID NO: 32 is a PCR primer for amplifying SEQ ID NO: 7.

SEQ ID NO: 33 is a PCR primer for amplifying SEQ ID NO: 8.

SEQ ID NO: 34 is a PCR primer for amplifying SEQ ID NO: 8.

SEQ ID NO: 35 is a PCR primer for amplifying SEQ ID NO: 9.

SEQ ID NO: 36 is a PCR primer for amplifying SEQ ID NO: 9.

SEQ ID NO: 37 is a PCR primer for amplifying SEQ ID NO: 10.

SEQ ID NO: 38 is a PCR primer for amplifying SEQ ID NO: 10.

SEQ ID NO: 39 is a PCR primer for amplifying SEQ ID NO: 11.

SEQ ID NO: 40 is a PCR primer for amplifying SEQ ID NO: 11.

SEQ ID NO: 41 is a PCR primer for amplifying SEQ ID NO: 12.

SEQ ID NO: 42 is a PCR primer for amplifying SEQ ID NO: 12.

SEQ ID NO: 43 is a PCR primer for amplifying SEQ ID NO: 13.

SEQ ID NO: 44 is a PCR primer for amplifying SEQ ID NO: 13.

SEQ ID NO: 45 is a PCR primer for amplifying SEQ ID NO: 14.

SEQ ID NO: 46 is a PCR primer for amplifying SEQ ID NO: 14.

SEQ ID NO: 47 is a PCR primer for amplifying SEQ ID NO: 15.

SEQ ID NO: 48 is a PCR primer for amplifying SEQ ID NO: 15.

SEQ ID NO: 49 is a PCR primer for amplifying SEQ ID NO: 16.

SEQ ID NO: 50 is a PCR primer for amplifying SEQ ID NO: 16.

SEQ ID NO: 51 is a PCR primer for amplifying SEQ ID NO: 17.

SEQ ID NO: 52 is a PCR primer for amplifying SEQ ID NO: 17.

SEQ ID NO: 53 is a PCR primer for amplifying SEQ ID NO: 18.

SEQ ID NO: 54 is a PCR primer for amplifying SEQ ID NO: 18.

SEQ ID NO: 55 is a first probe for detecting the genomic region associated with decreased α-subunit levels of SEQ ID NO: 1.

SEQ ID NO: 56 is a second probe for detecting the genomic region associated with decreased α-subunit levels of SEQ ID NO: 1.

SEQ ID NO: 57 is a first probe for detecting the genomic region associated with decreased α-subunit levels of SEQ ID NO: 2.

SEQ ID NO: 58 is a second probe for detecting the genomic region associated with decreased α-subunit levels of SEQ ID NO: 2.

SEQ ID NO: 59 is a first probe for detecting the genomic region associated with decreased α-subunit levels of SEQ ID NO: 3.

SEQ ID NO: 60 is a second probe for detecting the genomic region associated with decreased α-subunit levels of SEQ ID NO: 3.

SEQ ID NO: 61 is a first probe for detecting the genomic region associated with decreased α-subunit levels of SEQ ID NO: 4.

SEQ ID NO: 62 is a second probe for detecting the genomic region associated with decreased α-subunit levels of SEQ ID NO: 4.

SEQ ID NO: 63 is a first probe for detecting the genomic region associated with decreased α-subunit levels of SEQ ID NO: 5.

SEQ ID NO: 64 is a second probe for detecting the genomic region associated with decreased α-subunit levels of SEQ ID NO: 5.

SEQ ID NO: 65 is a first probe for detecting the genomic region associated with decreased α-subunit levels of SEQ ID NO: 6.

SEQ ID NO: 66 is a second probe for detecting the genomic region associated with decreased α-subunit levels of SEQ ID NO: 6.

SEQ ID NO: 67 is a first probe for detecting the genomic region associated with decreased α-subunit levels of SEQ ID NO: 7.

SEQ ID NO: 68 is a second probe for detecting the genomic region associated with decreased α-subunit levels of SEQ ID NO: 7.

SEQ ID NO: 69 is a first probe for detecting the genomic region associated with decreased α-subunit levels of SEQ ID NO: 8.

SEQ ID NO: 70 is a second probe for detecting the genomic region associated with decreased α-subunit levels of SEQ ID NO: 8.

SEQ ID NO: 71 is a first probe for detecting the genomic region associated with decreased α-subunit levels of SEQ ID NO: 9.

SEQ ID NO: 72 is a second probe for detecting the genomic region associated with decreased α-subunit levels of SEQ ID NO: 9.

SEQ ID NO: 73 is a first probe for detecting the genomic region associated with decreased α-subunit levels of SEQ ID NO: 10.

SEQ ID NO: 74 is a second probe for detecting the genomic region associated with decreased α-subunit levels of SEQ ID NO: 10.

SEQ ID NO: 75 is a first probe for detecting the genomic region associated with decreased α-subunit levels of SEQ ID NO: 11.

SEQ ID NO: 76 is a second probe for detecting the genomic region associated with decreased α-subunit levels of SEQ ID NO: 11.

SEQ ID NO: 77 is a first probe for detecting the genomic region associated with decreased α-subunit levels of SEQ ID NO: 12.

SEQ ID NO: 78 is a second probe for detecting the genomic region associated with decreased α-subunit levels of SEQ ID NO: 12.

SEQ ID NO: 79 is a first probe for detecting the genomic region associated with decreased α-subunit levels of SEQ ID NO: 13.

SEQ ID NO: 80 is a second probe for detecting the genomic region associated with decreased α-subunit levels of SEQ ID NO: 13.

SEQ ID NO: 81 is a first probe for detecting the genomic region associated with decreased α-subunit levels of SEQ ID NO: 14.

SEQ ID NO: 82 is a second probe for detecting the genomic region associated with decreased α-subunit levels of SEQ ID NO: 14.

SEQ ID NO: 83 is a first probe for detecting the genomic region associated with decreased α-subunit levels of SEQ ID NO: 15.

SEQ ID NO: 84 is a second probe for detecting the genomic region associated with decreased α-subunit levels of SEQ ID NO: 15.

SEQ ID NO: 85 is a first probe for detecting the genomic region associated with decreased α-subunit levels of SEQ ID NO: 16.

SEQ ID NO: 86 is a second probe for detecting the genomic region associated with decreased α-subunit levels of SEQ ID NO: 16.

SEQ ID NO: 87 is a first probe for detecting the genomic region associated with decreased α-subunit levels of SEQ ID NO: 17.

SEQ ID NO: 88 is a second probe for detecting the genomic region associated with decreased α-subunit levels of SEQ ID NO: 17.

SEQ ID NO: 89 is a first probe for detecting the genomic region associated with decreased α-subunit levels of SEQ ID NO: 18.

SEQ ID NO: 90 is a second probe for detecting the genomic region associated with decreased α-subunit levels of SEQ ID NO: 18.

### DESCRIPTION OF ILLUSTRATIVE EMBODIMENTS

The definitions and methods provided define the present invention and guide those of ordinary skill in the art in the practice of the present invention. Unless otherwise noted, terms are to be understood according to conventional usage by those of ordinary skill in the relevant art. Definitions of common terms in molecular biology may also be found in Alberts et al., Molecular Biology of The Cell, 3^{rd} Edition, Garland Publishing, Inc.: New York, 1994; Rieger et al., Glossary of Genetics: Classical and Molecular, 5th edition, Springer-Verlag: New York, 1991; and Lewin, Genes V, Oxford University Press: New York, 1994. The nomenclature for DNA bases as set forth at 37 CFR §1.822 is used.

The present invention provides plants and methods for producing plants comprising non-transgenic mutations that confer a seed β-conglycinin content comprising a decrease in α-subunit level, resulting in an increased α′-subunit level. Thus, plants of the invention are of great value as increased levels of α′-subunit of β-conglycinin provide improved nutritional characteristics and solubility of the soybean flour and protein isolates. Additionally, plants provided herein comprise agronomically elite characteristics, enabling a commercially significant yield.

The invention also provides plants and methods for producing plants comprising non-transgenic mutations that confer increased β-conglycinin and reduced glycinin. The combination of increased β-conglycinin and increased α′-subunit phenotype provides an increased content of the highly functional and healthful α′-subunit of β-conglycinin protein.

### I. PLANTS OF THE INVENTION

The invention provides, for the first time, plants and derivatives thereof of soybean that combine non-transgenic mutations conferring decreased α-subunit protein content resulting in increased α′-subunit content. In certain embodiments, the α′-subunit content of the seeds of plants of the invention may be greater than about 9, 10, 11, 12, 13, 14, 15, 16, 17, 18, 19 or even 20% of the total seed protein. In other embodiments, the glycinin content of the seeds of the plants of the invention maybe about or less than about 15, 14, 13, 12, 11, 10, 9, 8, 7, 6, 5, 4, 3, 2, 1, or 0 percent of the total seed protein, the β-conglycinin content of the seeds of the plants of the invention maybe about or at least about 34, 35, 36, 37, 38, 39, 40, 41, 42, 43, 44, 45, 46, 47, 48, 49, 50 percent or more of the total protein content, the α′-subunit content of the seeds of the plant of the invention maybe about or at least about 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 percent of total protein. In still further embodiments, a seed of the plant of the invention has β-conglycinin content comprising an α-subunit and an α′-subunit in a ratio of about 1.0, 0.9, 0.8, 0.7, 0.6, 0.5, 0.4, 0.3, 0.2, 0.1 or even 0.

One aspect of the current invention is therefore directed to the aforementioned plants and parts thereof and methods for using these plants and plant parts. Plant parts include, but are not limited to, pollen, an ovule and a cell. The invention further provides tissue cultures of regenerable cells of these plants, which cultures regenerate soybean plants capable of expressing all the physiological and morphological characteristics of the starting variety. Such regenerable cells may include embryos, meristematic cells, pollen, leaves, roots, root tips or flowers, or protoplasts or callus derived therefrom. Also provided by the invention are soybean plants regenerated from such a tissue culture, wherein the plants are capable of expressing all the physiological and morphological characteristics of the starting plant variety from which the regenerable cells were obtained.

### II. MARKER ASSISTED SELECTION FOR PRODUCTION OF SOYBEAN VARIETIES WITH NON-TRANSGENIC ALLELES THAT CONFER AN INCREASED β-CONGLYCININ α′-SUBUNIT AND DECREASED β-CONGLYCININ α-SUBUNIT CONTENT

The present invention describes methods to produce soybean plants with decreased α-subunit protein content resulting in increased α′-subunit protein content in seed. Moreover, the invention provides genetic markers and methods for the introduction of non-transgenic alleles that confer decreased α-subunit protein content resulting in increased β-conglycinin α′-subunit content into agronomically elite soybean plants. Certain aspects of the invention also provide methods for selecting parents for breeding of plants with decreased α-subunit protein content resulting in increased α′-subunit protein content in seed. One method involves screening germplasm for α′-subunit content in soybean seed. Another method includes identifying varieties which potentially carry the decreased α-subunit protein content resulting in increased α′-subunit trait by searching the pedigree of those varieties for presence of PI88788. The invention therefore allows, for the first time, the creation of plants that combine these alleles that confer increases α′-subunit seed content with a commercially significant yield and an agronomically elite genetic background. Using the methods of the invention, loci conferring the decreased α-subunit protein content resulting in increased α′-subunit may be introduced into a desired soybean genetic background, for example, in the production of new varieties with commercially significant yield and high seed β-conglycinin content.

The term quantitative trait loci, or QTL, is used to describe regions of a genome showing quantitative or additive effects upon a phenotype. The α′-subunit loci represent exemplary QTL since multiple α′-subunit alleles result in decreasing in total seed α-subunit content and important concomitant increases in α′-subunit content. Herein identified are genetic markers for non-transgenic, decreased α-subunit alleles resulting in increased α′-subunit content that enable breeding of soybean plants comprising the non-transgenic, decreased α-subunit alleles with agronomically superior plants, and selection of progeny that inherited the decreased α-subunit alleles. Thus, the invention allows the use of molecular tools to combine these QTLs with desired agronomic characteristics.

In the present invention, a decreased α-subunit protein content resulting in increased α′-subunit protein content locus is located on chromosome I. SNP markers used to monitor the introgression of locus include those selected from the group consisting of SEQ ID NO:1 through SEQ ID NO: 18. Illustrative locus SNP marker DNA sequences (SEQ ID NO: 1 through SEQ ID NO: 18) can be amplified using the primers indicated as SEQ ID NO: 19 through 54 with probes indicated as SEQ ID NO: 55 through 90.

The present invention also provides a soybean plant comprising a nucleic acid molecule selected from the group consisting of SEQ ID NO: 1 through SEQ ID NO: 18 and complements thereof. The present invention also provides a soybean plant comprising a nucleic acid molecule selected from the group consisting of SEQ ID NO: 1 through SEQ ID NOL 18, fragments thereof, and complements of both. The present invention also provides a soybean plant comprising a nucleic acid molecule selected from the group consisting of SEQ ID NO: 19 through SEQ ID NO: 90, fragments thereof, and complements of both. In one aspect, the soybean plant comprises 2, 3, 4, 5, 6, 7, 8, 9, 10, 11, 12, 13, 14, 15, 16, 17, or 18 nucleic acid molecules selected from the group consisting of SEQ ID NO: 1 through SEQ ID NO: 18 and complements thereof. In another aspect, the soybean plant comprises 2, 3, 4, 5, 6, 7, 8, 9, 10, 11, 12, 13, 14, 15, 16, 17, or 18 nucleic acid molecules selected from the group consisting of SEQ ID NO: 1 through SEQ ID NO: 18, fragments thereof, and complements of both. In a further aspect, the soybean plant comprises 2, 3, 4, 5, 6, 7, 8, 9, 10, 11, 12, 13, 14, 15, 16, 17, or 18 nucleic acid molecules selected from the group consisting of SEQ ID NO: 19 through SEQ ID NO: 90, fragments thereof, and complements of both.

The present invention also provides a soybean plant comprising a locus where one or more alleles at one or more of their loci are selected from the group consisting of allele 1, allele 2, allele 3, allele 4, allele 5, allele 6, allele 7, allele 8, allele 9, allele 10, allele 11, allele 12, allele 13, allele 14, allele 15, allele 16, allele 17 or allele 18. Such alleles may be homozygous or heterozygous.

Plants or parts thereof of the present invention may be grown in culture and regenerated. Methods for the regeneration of Glycine max plants from various tissue types and methods for the tissue culture of Glycine max are known in the art (See, for example, Widholm et al., In Vitro Selection and Culture-induced Variation in Soybean, In Soybean: Genetics, Molecular Biology and Biotechnology, Eds. Verma and Shoemaker, CAB International, Wallingford, Oxon, England (1996). Regeneration techniques for plants such as Glycine max can use as the starting material a variety of tissue or cell types. With Glycine max in particular, regeneration processes have been developed that begin with certain differentiated tissue types such as meristems, Cartha et al., Can. J. Bot. 59:1671-1679 (1981), hypocotyl sections, Cameya et al., Plant Science Letters 21: 289-294 (1981), and stem node segments, Saka et al., Plant Science Letters, 19: 193-201 (1980); Cheng et al., Plant Science Letters, 19: 91-99 (1980). Regeneration of whole sexually mature Glycine max plants from somatic embryos generated from explants of immature Glycine max embryos has been reported (Ranch et al., In Vitro Cellular & Developmental Biology 21: 653-658 (1985). Regeneration of mature Glycine max plants from tissue culture by organogenesis and embryogenesis has also been reported (Barwale et al., Planta 167: 473-481 (1986); Wright et al., Plant Cell Reports 5: 150-154 (1986).

The present invention also provides a plant with increased α′-subunit protein content in seed selected for by screening for seed protein content in the soybean plant, the selection comprising interrogating genomic nucleic acids for the presence of a marker molecule that is genetically linked to an allele of a QTL associated with increased α′-subunit protein content in seed of the soybean plant, where the allele of a QTL is also located on a linkage group associated with increased α′-subunit protein content in seed.

A method of introgressing an allele into a soybean plant comprising (A) crossing at least one first soybean plant comprising a nucleic acid molecule selected from the group consisting of SEQ ID NO: 1 through SEQ ID NO: 18 with at least one second soybean plant in order to form a segregating population, (B) screening the segregating population with one or more nucleic acid markers to determine if one or more soybean plants from the segregating population contains the nucleic acid molecule, and (C) selecting from the segregation population one or more soybean plants comprising a nucleic acid molecule selected from the group consisting of SEQ ID NO: 1 through SEQ ID NO: 18.

The present invention also includes a method of introgressing an allele into a soybean plant comprising: (A) crossing at least one soybean plant with decreased α-subunit protein content resulting in increased α′-subunit protein content in seed with at least second soybean plant in order to form a segregating population; (B) screening the segregating population with one or more nucleic acid markers to determine if one or more soybean plants from the segregating population contains an allele associated with decreased α-subunit protein content resulting in increased α′-subunit protein content in seed.

The present invention includes isolated nucleic acid molecules. Such molecules include those nucleic acid molecules capable of detecting a polymorphism genetically or physically linked to decreased α-subunit protein content resulting in an increased α′-subunit protein content in seed locus. Such molecules can be referred to as markers. Additional markers can be obtained that are linked to decreased α-subunit protein content resulting in an increased α′-subunit protein content in seed locus by available techniques. In one aspect, the nucleic acid molecule is capable of detecting the presence or absence of a marker located less than 30, 20, 10, 5, 2, or 1 centimorgans from a locus. In another aspect, a marker exhibits a LOD score of 2 or greater, 3 or greater, or 4 or greater, measuring using Qgene Version 2.23 (1996) and default parameters. In another aspect, the nucleic acid molecule is capable of detecting a marker in a locus. In a further aspect, a nucleic acid molecule is selected from the group consisting of SEQ ID NO: 1 through SEQ ID NO: 90, fragments thereof, complements thereof, and nucleic acid molecules capable of specifically hybridizing to one or more of these nucleic acid molecules.

In a preferred aspect, a nucleic acid molecule of the present invention includes those that will specifically hybridize to one or more of the nucleic acid molecules set forth in SEQ ID NO:1 through SEQ ID NO: 90 or complements thereof or fragments of either under moderately stringent conditions, for example at about 2.0×SSC and about 65° C. In a particularly preferred aspect, a nucleic acid of the present invention will specifically hybridize to one or more of the nucleic acid molecules set forth in SEQ ID NO: 1 through SEQ ID NO: 90 or complements or fragments of either under high stringency conditions. In one aspect of the present invention, a preferred marker nucleic acid molecule of the present invention has the nucleic acid sequence set forth in SEQ ID NO: 1 through SEQ ID NO: 90 or complements thereof or fragments of either. In another aspect of the present invention, a preferred marker nucleic acid molecule of the present invention shares between 80% and 100% or 90% and 100% sequence identity with the nucleic acid sequences set forth in SEQ ID NO: 1 through SEQ ID NO: 90 complements thereof or fragments of either. In a further aspect of the present invention, a preferred marker nucleic acid molecule of the present invention shares between 95% and 100% sequence identity with the sequences set forth in SEQ ID NO: 1 through SEQ ID NO: 90 or complements thereof or fragments of either. In a more preferred aspect of the present invention, a preferred marker nucleic acid molecule of the present invention shares between 98% and 100% sequence identity with the nucleic acid sequence set forth in SEQ ID NO: 1 through SEQ ID NO: 90 or complement thereof or fragments of either.

Nucleic acid molecules or fragments thereof are capable of specifically hybridizing to other nucleic acid molecules under certain circumstances. As used herein, two nucleic acid molecules are capable of specifically hybridizing to one another if the two molecules are capable of forming an anti-parallel, double-stranded nucleic acid structure. A nucleic acid molecule is the “complement” of another nucleic acid molecule if they exhibit complete complementarity. As used herein, molecules are exhibit “complete complementarity” when every nucleotide of one of the molecules is complementary to a nucleotide of the other. Two molecules are “minimally complementary” if they can hybridize to one another with sufficient stability to permit them to remain annealed to one another under at least conventional “low-stringency” conditions. Similarly, the molecules are “complementary” if they can hybridize to one another with sufficient stability to permit them to remain annealed to one another under conventional “high-stringency” conditions. Conventional stringency conditions are described by Sambrook et al., In: Molecular Cloning, A Laboratory Manual, 2nd Edition, Cold Spring Harbor Press, Cold Spring Harbor, N.Y. (1989), and by Haymes et al., In: Nucleic Acid Hybridization, A Practical Approach, IRL Press, Washington, D.C. (1985). Departures from complete complementarity are therefore permissible, as long as such departures do not completely preclude the capacity of the molecules to form a double-stranded structure. In order for a nucleic acid molecule to serve as a primer or probe it need only be sufficiently complementary in sequence to be able to form a stable double-stranded structure under the particular solvent and salt concentrations employed.

As used herein, a substantially homologous sequence is a nucleic acid sequence that will specifically hybridize to the complement of the nucleic acid sequence to which it is being compared under high stringency conditions. The nucleic-acid probes and primers of the present invention can hybridize under stringent conditions to a target DNA sequence. The term “stringent hybridization conditions” is defined as conditions under which a probe or primer hybridizes specifically with a target sequence(s) and not with non-target sequences, as can be determined empirically. The term “stringent conditions” is functionally defined with regard to the hybridization of a nucleic-acid probe to a target nucleic acid (i.e., to a particular nucleic-acid sequence of interest) by the specific hybridization procedure discussed in Sambrook et al., 1989, at 9.52-9.55. See also, Sambrook et al., 1989 at 9.47-9.52, 9.56-9.58; Kanehisa 1984 Nucl. Acids Res. 12:203-213; and Wetmur et al. 1968 J. Mol. Biol. 31:349-370. Appropriate stringency conditions that promote DNA hybridization are, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y., 1989, 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed.

For example, hybridization using DNA or RNA probes or primers can be performed at 65° C. in 6×SSC, 0.5% SDS, 5×Denhardt's, 100 μg/mL nonspecific DNA (e.g., sonicated salmon sperm DNA) with washing at 0.5×SSC, 0.5% SDS at 65° C., for high stringency.

It is contemplated that lower stringency hybridization conditions such as lower hybridization and/or washing temperatures can be used to identify related sequences having a lower degree of sequence similarity if specificity of binding of the probe or primer to target sequence(s) is preserved. Accordingly, the nucleotide sequences of the present invention can be used for their ability to selectively form duplex molecules with complementary stretches of DNA, RNA, or cDNA fragments.

A fragment of a nucleic acid molecule can be any sized fragment and illustrative fragments include fragments of nucleic acid sequences set forth in SEQ ID NO: 1 through SEQ ID NO: 90 and complements thereof. In one aspect, a fragment can be between 15 and 25, 15 and 30, 15 and 40, 15 and 50, 15 and 100, 20 and 25, 20 and 30, 20 and 40, 20 and 50, 20 and 100, 25 and 30, 25 and 40, 25 and 50, 25 and 100, 30 and 40, 30 and 50, and 30 and 100. In another aspect, the fragment can be greater than 10, 15, 20, 25, 30, 35, 40, 50, 100, or 250 nucleotides.

Additional genetic markers can be used to select plants with an allele of a QTL associated with reduce α-subunit protein content resulting in increased α′-subunit protein content in seed of the soybean plant of the present invention. Examples of public marker databases include, for example: Soybase, an Agricultural Research Service, United States Department of Agriculture.

Genetic markers of the present invention include “dominant” or “codominant” markers. “Codominant markers” reveal the presence of two or more alleles (two per diploid individual). “Dominant markers” reveal the presence of only a single allele. The presence of the dominant marker phenotype (e.g., a band of DNA) is an indication that one allele is present in either the homozygous or heterozygous condition. The absence of the dominant marker phenotype (e.g., absence of a DNA band) is merely evidence that “some other” undefined allele is present. In the case of populations where individuals are predominantly homozygous and loci are predominantly dimorphic, dominant and codominant markers can be equally valuable. As populations become more heterozygous and multiallelic, codominant markers often become more informative of the genotype than dominant markers.

In another embodiment, markers, such as single sequence repeat markers (SSR), AFLP markers, RFLP markers, RAPD markers, phenotypic markers, isozyme markers, single nucleotide polymorphisms (SNPs), insertions or deletions (Indels), single feature polymorphisms (SFPs, for example, as described in Borevitz et al. 2003 Gen. Res. 13:513-523), microarray transcription profiles, DNA-derived sequences, and RNA-derived sequences that are genetically linked to or correlated with alleles of a QTL of the present invention can be utilized.

In one embodiment, nucleic acid-based analyses for the presence or absence of the genetic polymorphism can be used for the selection of seeds in a breeding population. A wide variety of genetic markers for the analysis of genetic polymorphisms are available and known to those of skill in the art. The analysis may be used to select for genes, QTL, alleles, or genomic regions (haplotypes) that comprise or are linked to a genetic marker.

Herein, nucleic acid analysis methods are known in the art and include, but are not limited to, PCR-based detection methods (for example, TaqMan assays), microarray methods, and nucleic acid sequencing methods. In one embodiment, the detection of polymorphic sites in a sample of DNA, RNA, or cDNA may be facilitated through the use of nucleic acid amplification methods. Such methods specifically increase the concentration of polynucleotides that span the polymorphic site, or include that site and sequences located either distal or proximal to it. Such amplified molecules can be readily detected by gel electrophoresis, fluorescence detection methods, or other means.

A method of achieving such amplification employs the polymerase chain reaction (PCR) (Mullis et al. 1986 Cold Spring Harbor Symp. Quant. Biol. 51:263-273; European Patent 50,424; European Patent 84,796; European Patent 258,017; European Patent 237,362; European Patent 201,184; U.S. Pat. No. 4,683,202; U.S. Pat. No. 4,582,788; and U.S. Pat. No. 4,683,194), using primer pairs that are capable of hybridizing to the proximal sequences that define a polymorphism in its double-stranded form

Polymorphisms in DNA sequences can be detected or typed by a variety of effective methods well known in the art including, but not limited to, those disclosed in U.S. Pat. Nos. 5,468,613 and 5,217,863; 5,210,015; 5,876,930; 6,030,787; 6,004,744; 6,013,431; 5,595,890; 5,762,876; 5,945,283; 5,468,613; 6,090,558; 5,800,944; and 5,616,464, all of which are incorporated herein by reference in their entireties. However, the compositions and methods of this invention can be used in conjunction with any polymorphism typing method to type polymorphisms in soybean genomic DNA samples. These soybean genomic DNA samples used include but are not limited to soybean genomic DNA isolated directly from a soybean plant, cloned soybean genomic DNA, or amplified soybean genomic DNA.

For instance, polymorphisms in DNA sequences can be detected by hybridization to allele-specific oligonucleotide (ASO) probes as disclosed in U.S. Pat. Nos. 5,468,613 and 5,217,863. U.S. Pat. No. 5,468,613 discloses allele specific oligonucleotide hybridizations where single or multiple nucleotide variations in nucleic acid sequence can be detected in nucleic acids by a process in which the sequence containing the nucleotide variation is amplified, spotted on a membrane and treated with a labeled sequence-specific oligonucleotide probe.

Target nucleic acid sequence can also be detected by probe ligation methods as disclosed in U.S. Pat. No. 5,800,944 where sequence of interest is amplified and hybridized to probes followed by ligation to detect a labeled part of the probe.

Microarrays can also be used for polymorphism detection, wherein oligonucleotide probe sets are assembled in an overlapping fashion to represent a single sequence such that a difference in the target sequence at one point would result in partial probe hybridization (Borevitz et al., Genome Res. 13:513-523 (2003); Cui et al., Bioinformatics 21:3852-3858 (2005). On any one microarray, it is expected there will be a plurality of target sequences, which may represent genes and/or noncoding regions wherein each target sequence is represented by a series of overlapping oligonucleotides, rather than by a single probe. This platform provides for high throughput screening a plurality of polymorphisms. A single-feature polymorphism (SFP) is a polymorphism detected by a single probe in an oligonucleotide array, wherein a feature is a probe in the array. Typing of target sequences by microarray-based methods is disclosed in U.S. Pat. Nos. 6,799,122; 6,913,879; and 6,996,476.

Target nucleic acid sequence can also be detected by probe linking methods as disclosed in U.S. Pat. No. 5,616,464 employing at least one pair of probes having sequences homologous to adjacent portions of the target nucleic acid sequence and having side chains which non-covalently bind to form a stem upon base pairing of said probes to said target nucleic acid sequence. At least one of the side chains has a photoactivatable group which can form a covalent cross-link with the other side chain member of the stem.

Other methods for detecting SNPs and Indels include single base extension (SBE) methods. Examples of SBE methods include, but are not limited, to those disclosed in U.S. Pat. Nos. 6,004,744; 6,013,431; 5,595,890; 5,762,876; and 5,945,283. SBE methods are based on extension of a nucleotide primer that is immediately adjacent to a polymorphism to incorporate a detectable nucleotide residue upon extension of the primer. In certain embodiments, the SBE method uses three synthetic oligonucleotides. Two of the oligonucleotides serve as PCR primers and are complementary to sequence of the locus of soybean genomic DNA which flanks a region containing the polymorphism to be assayed. Following amplification of the region of the soybean genome containing the polymorphism, the PCR product is mixed with the third oligonucleotide (called an extension primer) which is designed to hybridize to the amplified DNA immediately adjacent to the polymorphism in the presence of DNA polymerase and two differentially labeled dideoxynucleosidetriphosphates. If the polymorphism is present on the template, one of the labeled dideoxynucleosidetriphosphates can be added to the primer in a single base chain extension. The allele present is then inferred by determining which of the two differential labels was added to the extension primer. Homozygous samples will result in only one of the two labeled bases being incorporated and thus only one of the two labels will be detected. Heterozygous samples have both alleles present, and will thus direct incorporation of both labels (into different molecules of the extension primer) and thus both labels will be detected.

In a preferred method for detecting polymorphisms, SNPs and Indels can be detected by methods disclosed in U.S. Pat. Nos. 5,210,015; 5,876,930; and 6,030,787 in which an oligonucleotide probe having a 5′fluorescent reporter dye and a 3′quencher dye covalently linked to the 5′ and 3′ ends of the probe. When the probe is intact, the proximity of the reporter dye to the quencher dye results in the suppression of the reporter dye fluorescence, e.g. by Forster-type energy transfer. During PCR forward and reverse primers hybridize to a specific sequence of the target DNA flanking a polymorphism while the hybridization probe hybridizes to polymorphism-containing sequence within the amplified PCR product. In the subsequent PCR cycle DNA polymerase with 5′→3′ exonuclease activity cleaves the probe and separates the reporter dye from the quencher dye resulting in increased fluorescence of the reporter.

For the purpose of QTL mapping, the markers included should be diagnostic of origin in order for inferences to be made about subsequent populations. SNP markers are ideal for mapping because the likelihood that a particular SNP allele is derived from independent origins in the extant populations of a particular species is very low. As such, SNP markers are useful for tracking and assisting introgression of QTLs, particularly in the case of haplotypes.

The genetic linkage of additional marker molecules can be established by a gene mapping model such as, without limitation, the flanking marker model reported by Lander et al. (Lander et al. 1989 Genetics, 121:185-199), and the interval mapping, based on maximum likelihood methods described therein, and implemented in the software package MAPMAKER/QTL (Lincoln and Lander, Mapping Genes Controlling Quantitative Traits Using MAPMAKER/QTL, Whitehead Institute for Biomedical Research, Massachusetts, (1990). Additional software includes Qgene, Version 2.23 (1996), Department of Plant Breeding and Biometry, 266 Emerson Hall, Cornell University, Ithaca, N.Y.). Use of Qgene software is a particularly preferred approach.

A maximum likelihood estimate (MLE) for the presence of a marker is calculated, together with an MLE assuming no QTL effect, to avoid false positives. A log₁₀ of an odds ratio (LOD) is then calculated as: LOD=log₁₀ (MLE for the presence of a QTL/MLE given no linked QTL). The LOD score essentially indicates how much more likely the data are to have arisen assuming the presence of a QTL versus in its absence. The LOD threshold value for avoiding a false positive with a given confidence, say 95%, depends on the number of markers and the length of the genome. Graphs indicating LOD thresholds are set forth in Lander et al. (1989), and further described by Arús and Moreno-González, Plant Breeding, Hayward, Bosemark, Romagosa (eds.) Chapman & Hall, London, pp. 314-331 (1993).

Additional models can be used. Many modifications and alternative approaches to interval mapping have been reported, including the use of non-parametric methods (Kruglyak et al. 1995 Genetics, 139:1421-1428). Multiple regression methods or models can be also be used, in which the trait is regressed on a large number of markers (Jansen, Biometrics in Plant Breed, van Oijen, Jansen (eds.) Proceedings of the Ninth Meeting of the Eucarpia Section Biometrics in Plant Breeding, The Netherlands, pp. 116-124 (1994); Weber and Wricke, Advances in Plant Breeding, Blackwell, Berlin, 16 (1994)). Procedures combining interval mapping with regression analysis, whereby the phenotype is regressed onto a single putative QTL at a given marker interval, and at the same time onto a number of markers that serve as ‘cofactors,’ have been reported by Jansen et al. (Jansen et al. 1994 Genetics, 136:1447-1455) and Zeng (Zeng 1994 Genetics 136:1457-1468). Generally, the use of cofactors reduces the bias and sampling error of the estimated QTL positions (Utz and Melchinger, Biometrics in Plant Breeding, van Oijen, Jansen (eds.) Proceedings of the Ninth Meeting of the Eucarpia Section Biometrics in Plant Breeding, The Netherlands, pp. 195-204 (1994), thereby improving the precision and efficiency of QTL mapping (Zeng 1994). These models can be extended to multi-environment experiments to analyze genotype-environment interactions (Jansen et al. 1995 Theor. Appl. Genet. 91:33-3).

Selection of appropriate mapping populations is important to map construction. The choice of an appropriate mapping population depends on the type of marker systems employed (Tanksley et al., Molecular mapping in plant chromosomes, chromosome structure and function: Impact of new concepts J. P. Gustafson and R. Appels (eds.). Plenum Press, New York, pp. 157-173 (1988)). Consideration must be given to the source of parents (adapted vs. exotic) used in the mapping population. Chromosome pairing and recombination rates can be severely disturbed (suppressed) in wide crosses (adapted×exotic) and generally yield greatly reduced linkage distances. Wide crosses will usually provide segregating populations with a relatively large array of polymorphisms when compared to progeny in a narrow cross (adapted×adapted).

Marker assisted introgression involves the transfer of a chromosome region defined by one or more markers from one germplasm to a second germplasm. The initial step in that process is the localization of the trait by gene mapping, which is the process of determining the position of a gene relative to other genes and genetic markers through linkage analysis. The basic principle for linkage mapping is that the closer together two genes are on the chromosome, the more likely they are to be inherited together. Briefly, a cross is generally made between two genetically compatible but divergent parents relative to traits under study. Genetic markers can then be used to follow the segregation of traits under study in the progeny from the cross, often a backcross (BC1), F₂, or recombinant inbred population.

A. Development and Use of Linked Genetic Markers

A sample first plant population may be genotyped for an inherited genetic marker to form a genotypic database. As used herein, an “inherited genetic marker” is an allele at a single locus. A locus is a position on a chromosome, and allele refers to conditions of genes; that is, different nucleotide sequences, at those loci. The marker allelic composition of each locus can be either homozygous or heterozygous. In order for information to be gained from a genetic marker in a cross, the marker must be polymorphic; that is, it must exist in different forms so that the chromosome carrying the mutant gene can be distinguished from the chromosome with the normal gene by the form of the marker it also carries.

Formation of a phenotypic database can be accomplished by making direct observations of one or more traits on progeny derived from artificial or natural self-pollination of a sample plant or by quantitatively assessing the combining ability of a sample plant. By way of example, a plant line may be crossed to, or by, one or more testers. Testers can be inbred lines, single, double, or multiple cross hybrids, or any other assemblage of plants produced or maintained by controlled or free mating, or any combination thereof. For some self-pollinating plants, direct evaluation without progeny testing is preferred.

The marker genotypes may be determined in the testcross generation and the marker loci mapped. To map a particular trait by the linkage approach, it is necessary to establish a positive correlation in inheritance of a specific chromosomal locus with the inheritance of the trait. In the case of complex inheritance, such as with quantitative traits, including specifically α-subunit content and yield, linkage will generally be much more difficult to discern. In this case, statistical procedures may be needed to establish the correlation between phenotype and genotype. This may further necessitate examination of many offspring from a particular cross, as individual loci may have small contributions to an overall phenotype.

Coinheritance, or genetic linkage, of a particular trait and a marker suggests that they are physically close together on the chromosome. Linkage is determined by analyzing the pattern of inheritance of a gene and a marker in a cross. The unit of genetic map distance is the centimorgan (cM), which increases with increasing recombination. Two markers are one centimorgan apart if they recombine in meiosis about once in every 100 opportunities that they have to do so. The centimorgan is a genetic measure, not a physical one. Those markers located less then 50 cM from a second locus are said to be genetically linked, because they are not inherited independently of one another. Thus, the percent of recombination observed between the loci per generation will be less than 50%. In particular embodiments of the invention, a marker used may be defined as located less than about 45, 35, 25, 15, 10, 5, 4, 3, 2, or 1 or less cM apart from a locus.

During meiosis, pairs of homologous chromosomes come together and exchange segments in a process called recombination. The further a marker is from a gene, the more chance there is that there will be recombination between the gene and the marker. In a linkage analysis, the coinheritance of marker and gene or trait are followed in a particular cross. The probability that their observed inheritance pattern could occur by chance alone, i.e., that they are completely unlinked, is calculated. The calculation is then repeated assuming a particular degree of linkage, and the ratio of the two probabilities (no linkage versus a specified degree of linkage) is determined. This ratio expresses the odds for (and against) that degree of linkage, and because the logarithm of the ratio is used, it is known as the logarithm of the odds, e.g. an lod score. A lod score equal to or greater than 3, for example, is taken to confirm that gene and marker are linked. This represents 1000:1 odds that the two loci are linked Calculations of linkage is greatly facilitated by use of statistical analysis employing programs.

The genetic linkage of marker molecules can be established by a gene mapping model such as, without limitation, the flanking marker model reported by Lander and Botstein (1989), and the interval mapping, based on maximum likelihood methods described by Lander and Botstein (1989), and implemented in the software package MAPMAKER/QTL. Additional software includes Qgene, Version 2.23 (1996) (Department of Plant Breeding and Biometry, 266 Emerson Hall, Cornell University, Ithaca, N.Y.).

B. Inherited Markers

Genetic markers comprise detected differences (polymorphisms) in the genetic information carried by two or more plants. Genetic mapping of a locus with genetic markers typically requires two fundamental components: detectably polymorphic alleles and recombination or segregation of those alleles. In plants, the recombination measured is virtually always meiotic, and therefore, the two inherent requirements of plant gene mapping are polymorphic genetic markers and one or more plants in which those alleles are segregating.

Markers are preferably inherited in codominant fashion so that the presence of both alleles at a diploid locus is readily detectable, and they are free of environmental variation, i.e., their heritability is 1. A marker genotype typically comprises two marker alleles at each locus in a diploid organism such as soybeans. The marker allelic composition of each locus can be either homozygous or heterozygous. Homozygosity is a condition where both alleles at a locus are characterized by the same nucleotide sequence. Heterozygosity refers to different conditions of the gene at a locus.

A number of different marker types are available for use in genetic mapping. Exemplary genetic marker types for use with the invention include, but are not limited to, restriction fragment length polymorphisms (RFLPs), simple sequence length polymorphisms (SSLPs), amplified fragment length polymorphisms (AFLPs), single nucleotide polymorphisms (SNPs), nucleotide insertions and/or deletions (INDELs) and isozymes. Polymorphisms comprising as little as a single nucleotide change can be assayed in a number of ways. For example, detection can be made by electrophoretic techniques including a single strand conformational polymorphism (Orita et al., 1989), denaturing gradient gel electrophoresis (Myers et al., 1985), or cleavage fragment length polymorphisms (Life Technologies, Inc., Gathersberg, Md. 20877), but the widespread availability of DNA sequencing machines often makes it easier to just sequence amplified products directly. Once the polymorphic sequence difference is known, rapid assays can be designed for progeny testing, typically involving some version of PCR amplification of specific alleles (PASA, Sommer, et al., 1992), or PCR amplification of multiple specific alleles (PAMSA, Dutton and Sommer, 1991). The analysis may be used to select for genes, QTL, alleles, or genomic regions (haplotypes) that comprise or are linked to a genetic marker.

Nucleic acid analysis methods are known in the art and include, but are not limited to, PCR-based detection methods (for example, TaqMan assays), microarray methods, and nucleic acid sequencing methods. The detection of polymorphic sites in a sample of DNA, RNA, or cDNA may be facilitated through the use of nucleic acid amplification methods.

One method for detection of SNPs in DNA, RNA and cDNA samples is by use of PCR in combination with fluorescent probes for the polymorphism, as described in Livak et al., 1995 and U.S. Pat. No. 5,604,099, incorporated herein by reference. Such methods specifically increase the concentration of polynucleotides that span the polymorphic site, or include that site and sequences located either distal or proximal to it. Such amplified molecules can be readily detected by gel electrophoresis, fluorescence detection methods, or other means. Briefly, probe oligonucleotides, one of which anneals to the SNP site and the other which anneals to the wild type sequence, are synthesized. It is preferable that the site of the SNP be near the 5′ terminus of the probe oligonucleotides. Each probe is then labeled on the 3′ end with a non-fluorescent quencher and a minor groove binding moiety which lower background fluorescence and lower the Tₘ of the oligonucleotide, respectively. The 5′ ends of each probe are labeled with a different fluorescent dye wherein fluorescence is dependent upon the dye being cleaved from the probe. Some non-limiting examples of such dyes include VIC™ and 6-FAM™. DNA suspected of comprising a given SNP is then subjected to PCR using a polymerase with 5′-3′ exonuclease activity and flanking primers. PCR is performed in the presence of both probe oligonucleotides. If the probe is bound to a complimentary sequence in the test DNA then exonuclease activity of the polymerase releases a fluorescent label activating its fluorescent activity. Therefore, test DNA that contains only wild type sequence will exhibit fluorescence associated with the label on the wild type probe. On the other hand, DNA containing only the SNP sequence will have fluorescent activity from the label on the SNP probe. However, in the case that the DNA is from heterogeneous sources, significant fluorescence of both labels will be observed. This type of indirect genotyping at known SNP sites enables high throughput, inexpensive screening of DNA samples. Thus such a system is ideal for the identification of progeny soybean plants comprising α′-subunit alleles.

Restriction fragment length polymorphisms (RFLPs) are genetic differences detectable by DNA fragment lengths, typically revealed by agarose gel electrophoresis, after restriction endonuclease digestion of DNA. There are large numbers of restriction endonucleases available, characterized by their nucleotide cleavage sites and their source, e.g., EcoRI. RFLPs result from both single-bp polymorphisms within restriction site sequences and measurable insertions or deletions within a given restriction fragment. RFLPs are easy and relatively inexpensive to generate (require a cloned DNA, but no sequence) and are co-dominant. RFLPs have the disadvantage of being labor-intensive in the typing stage, although this can be alleviated to some extent by multiplexing many of the tasks and reutilization of blots. Most RFLP are biallelic and of lesser polymorphic content than microsatellites. For these reasons, the use of RFLP in plant genetic maps has waned.

One of skill in the art would recognize that many types of molecular markers are useful as tools to monitor genetic inheritance and are not limited to RFLPs, SSRs and SNPs, and one of skill would also understand that a variety of detection methods may be employed to track the various molecular markers. One skilled in the art would also recognize that markers of different types may be used for mapping, especially as technology evolves and new types of markers and means for identification are identified.

For purposes of convenience, inherited marker genotypes may be converted to numerical scores, e.g., if there are 2 forms of an SNP, or other marker, designated A and B, at a particular locus using a particular enzyme, then diploid complements may be converted to a numerical score, for example, are AA=2, AB=1, and BB=0; or AA=1, AB=0 and BB=−1. The absolute values of the scores are not important. What is important is the additive nature of the numeric designations. The above scores relate to codominant markers. A similar scoring system can be given that is consistent with dominant markers.

C. Marker Assisted Selection

The invention provides soybean plants with increased β-conglycinin content in combination with a commercially significant yield and agronomically elite characteristics. Such plants may be produced in accordance with the invention by marker assisted selection methods comprising assaying genomic DNA for the presence of markers that are genetically linked to the non-transgenic, α-subunit allele 1 through allele 18, including all possible combinations thereof.

In certain embodiments of the invention, it may be desired to obtain additional markers linked to α-subunit alleles. This may be carried out, for example, by first preparing an F₂ population by selfing an F₁ hybrid produced by crossing inbred varieties only one of which comprises α-subunit allele conferring a decrease α-subunit content resulting in increased α′-subunit content. Recombinant inbred lines (RIL) (genetically related lines; usually >F₅, developed from continuously selfing F₂ lines towards homozygosity) can then be prepared and used as a mapping population. Information obtained from dominant markers can be maximized by using RIL because all loci are homozygous or nearly so.

Backcross populations (e.g., generated from a cross between a desirable variety (recurrent parent) and another variety (donor parent)) carrying a trait not present in the former can also be utilized as a mapping population. A series of backcrosses to the recurrent parent can be made to recover most of its desirable traits. Thus a population is created consisting of individuals similar to the recurrent parent but each individual carries varying amounts of genomic regions from the donor parent. Backcross populations can be useful for mapping dominant markers if all loci in the recurrent parent are homozygous and the donor and recurrent parent have contrasting polymorphic marker alleles (Reiter et al., 1992).

Useful populations for mapping purposes are near-isogenic lines (NIL). NILs are created by many backcrosses to produce an array of individuals that are nearly identical in genetic composition except for the desired trait or genomic region can be used as a mapping population. In mapping with NILs, only a portion of the polymorphic loci are expected to map to a selected region. Mapping may also be carried out on transformed plant lines.

D. Plant Breeding Methods

Certain aspects of the invention provide methods for marker assisted breeding of plants that enable the introduction of non-transgenic, α-subunit alleles into a heterologous soybean genetic background. In general, breeding techniques take advantage of a plant's method of pollination. There are two general methods of pollination: self-pollination which occurs if pollen from one flower is transferred to the same or another flower of the same plant, and cross-pollination which occurs if pollen comes to it from a flower on a different plant. Plants that have been self-pollinated and selected for type over many generations become homozygous at almost all gene loci and produce a uniform population of true breeding progeny, homozygous plants.

In development of suitable varieties, pedigree breeding may be used. The pedigree breeding method for specific traits involves crossing two genotypes. Each genotype can have one or more desirable characteristics lacking in the other; or, each genotype can complement the other. If the two original parental genotypes do not provide all of the desired characteristics, other genotypes can be included in the breeding population. Superior plants that are the products of these crosses are selfed and are again advanced in each successive generation. Each succeeding generation becomes more homogeneous as a result of self-pollination and selection. Typically, this method of breeding involves five or more generations of selfing and selection: S₁→S₂; S₂→S₃; S₃→S₄; S₄→S₅, etc. A selfed generation (S) may be considered to be a type of filial generation (F) and may be named F as such. After at least five generations, the inbred plant is considered genetically pure.

Each breeding program should include a periodic, objective evaluation of the efficiency of the breeding procedure. Evaluation criteria vary depending on the goal and objectives. Promising advanced breeding lines are thoroughly tested and compared to appropriate standards in environments representative of the commercial target area(s) for generally three or more years. Identification of individuals that are genetically superior is difficult because genotypic value can be masked by confounding plant traits or environmental factors. One method of identifying a superior plant is to observe its performance relative to other experimental plants and to one or more widely grown standard varieties. Single observations can be inconclusive, while replicated observations provide a better estimate of genetic worth.

Mass and recurrent selections can be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued. Descriptions of other breeding methods that are commonly used for different traits and crops can be found in one of several reference books (e.g., Allard, 1960; Simmonds, 1979; Sneep et al., 1979; Fehr, 1987a,b).

The effectiveness of selecting for genotypes with traits of interest (e.g., high yield, disease resistance, fatty acid profile) in a breeding program will depend upon: 1) the extent to which the variability in the traits of interest of individual plants in a population is the result of genetic factors and is thus transmitted to the progenies of the selected genotypes; and 2) how much the variability in the traits of interest among the plants is due to the environment in which the different genotypes are growing. The inheritance of traits ranges from control by one major gene whose expression is not influenced by the environment (i.e., qualitative characters) to control by many genes whose effects are greatly influenced by the environment (i.e., quantitative characters). Breeding for quantitative traits such as yield is further characterized by the fact that: 1) the differences resulting from the effect of each gene are small, making it difficult or impossible to identify them individually; 2) the number of genes contributing to a character is large, so that distinct segregation ratios are seldom if ever obtained; and 3) the effects of the genes may be expressed in different ways based on environmental variation. Therefore, the accurate identification of transgressive segregates or superior genotypes with the traits of interest is extremely difficult and its success is dependent on the plant breeder's ability to minimize the environmental variation affecting the expression of the quantitative character in the population.

The likelihood of identifying a transgressive segregant is greatly reduced as the number of traits combined into one genotype is increased. For example, if a cross is made between cultivars differing in three complex characters, such as yield, α′-subunit content and at least a first agronomic trait, it is extremely difficult without molecular tools to recover simultaneously by recombination the maximum number of favorable genes for each of the three characters into one genotype. Consequently, all the breeder can generally hope for is to obtain a favorable assortment of genes for the first complex character combined with a favorable assortment of genes for the second character into one genotype in addition to a selected gene.

Backcrossing is an efficient method for transferring specific desirable traits. This can be accomplished, for example, by first crossing a superior variety inbred (A) (recurrent parent) to a donor inbred (non-recurrent parent), which carries the appropriate gene(s) for the trait in question (Fehr, 1987). The progeny of this cross are then mated back to the superior recurrent parent (A) followed by selection in the resultant progeny for the desired trait to be transferred from the non-recurrent parent. Such selection can be based on genetic assays, as mentioned below, or alternatively, can be based on the phenotype of the progeny plant. After five or more backcross generations with selection for the desired trait, the progeny are heterozygous for loci controlling the characteristic being transferred, but are like the superior parent for most or almost all other genes. The last generation of the backcross is selfed, or sibbed, to give pure breeding progeny for the gene(s) being transferred, for example, loci providing the plant with decreased seed glycinin content.

In one embodiment of the invention, the process of backcross conversion may be defined as a process including the steps of:
(a) crossing a plant of a first genotype containing one or more desired gene, DNA sequence or element, such as α-subunit allele 1 through α-subunit allele 18 associated with increased seed α-subunit content, to a plant of a second genotype lacking said desired gene, DNA sequence or element;
(b) selecting one or more progeny plant(s) containing the desired gene, DNA sequence or element;
(c) crossing the progeny plant to a plant of the second genotype; and
(d) repeating steps (b) and (c) for the purpose of transferring said desired gene, DNA sequence or element from a plant of a first genotype to a plant of a second genotype.

Introgression of a particular DNA element or set of elements into a plant genotype is defined as the result of the process of backcross conversion. A plant genotype into which a DNA sequence has been introgressed may be referred to as a backcross converted genotype, line, inbred, or hybrid. Similarly a plant genotype lacking the desired DNA sequence may be referred to as an unconverted genotype, line, inbred, or hybrid. During breeding, the genetic markers linked to decrease α-subunit content resulting increased α′-subunit content may be used to assist in breeding for the purpose of producing soybean plants with increased α′-subunit content. Backcrossing and marker assisted selection in particular can be used with the present invention to introduce the increased α′-subunit content trait in accordance with the current invention into any variety by conversion of that variety with non-transgenic α′-subunit allele 1 through allele 18 associated.

The selection of a suitable recurrent parent is an important step for a successful backcrossing procedure. The goal of a backcross protocol is to alter or substitute a trait or characteristic in the original inbred. To accomplish this, one or more loci of the recurrent inbred is modified or substituted with the desired gene from the nonrecurrent parent, while retaining essentially all of the rest of the desired genetic, and therefore the desired physiological and morphological, constitution of the original inbred. The choice of the particular nonrecurrent parent will depend on the purpose of the backcross, which in the case of the present invention may be to add one or more allele(s) conferring increased α′-subunit content. The exact backcrossing protocol will depend on the characteristic or trait being altered to determine an appropriate testing protocol. Although backcrossing methods are simplified when the characteristic being transferred is a dominant allele, a recessive allele may also be transferred. In this instance it may be necessary to introduce a test of the progeny to determine if the desired characteristic has been successfully transferred. In the case of the present invention, one may test the glycinin content of progeny lines generated during the backcrossing program, for example by SDS-PAGE/Coomassie staining as well as using the marker system described herein to select lines based upon markers rather than visual traits.

Soybean plants (Glycine max L.) can be crossed by either natural or mechanical techniques (see, e.g., Fehr, In: Hybridization of Crop Plants, Fehr and Hadley (Eds.), Am. Soc. Agron. and Crop Sci. Soc. Am., Madison, Wis., 90-599 (1980). Natural pollination occurs in soybeans either by self pollination or natural cross pollination, which typically is aided by pollinating organisms. In either natural or artificial crosses, flowering and flowering time are an important consideration. Soybean is a short-day plant, but there is considerable genetic variation for sensitivity to photoperiod (Hamner, 1969; Criswell and Hume, 1972). The critical day length for flowering ranges from about 13 h for genotypes adapted to tropical latitudes to 24 h for photoperiod-insensitive genotypes grown at higher latitudes (Shibles et al., 1975). Soybeans seem to be insensitive to day length for 9 days after emergence. Photoperiods shorter than the critical day length are required for 7 to 26 days to complete flower induction (Borthwick and Parker, 1938; Shanmugasundaram and Tsou, 1978).

Either with or without emasculation of the female flower, hand pollination can be carried out by removing the stamens and pistil with a forceps from a flower of the male parent and gently brushing the anthers against the stigma of the female flower. Access to the stamens can be achieved by removing the front sepal and keel petals, or piercing the keel with closed forceps and allowing them to open to push the petals away. Brushing the anthers on the stigma causes them to rupture, and the highest percentage of successful crosses is obtained when pollen is clearly visible on the stigma. Pollen shed can be checked by tapping the anthers before brushing the stigma. Several male flowers may have to be used to obtain suitable pollen shed when conditions are unfavorable, or the same male may be used to pollinate several flowers with good pollen shed.

Genetic male sterility is available in soybeans and may be useful to facilitate hybridization in the context of the current invention, particularly for recurrent selection programs (Brim and Stuber, 1973). The distance required for complete isolation of a crossing block is not clear; however, outcrossing is less than 0.5% when male-sterile plants are 12 m or more from a foreign pollen source (Boerma and Moradshahi, 1975). Plants on the boundaries of a crossing block probably sustain the most outcrossing with foreign pollen and can be eliminated at harvest to minimize contamination.

Once harvested, pods are typically air-dried at not more than 38° C. until the seeds contain 13% moisture or less, then the seeds are removed by hand. Seed can be stored satisfactorily at about 25° C. for up to a year if relative humidity is 50% or less. In humid climates, germination percentage declines rapidly unless the seed is dried to 7% moisture and stored in an air-tight container at room temperature. Long-term storage in any climate is best accomplished by drying seed to 7% moisture and storing it at 10° C. or less in a room maintained at 50% relative humidity or in an air-tight container.

### III. TRAITS FOR MODIFICATION AND IMPROVEMENT OF SOYBEAN VARIETIES

In certain embodiments, a soybean plant provided by the invention may comprise one or more transgene(s). One example of such a transgene confers herbicide resistance. Common herbicide resistance genes include an EPSPS gene conferring glyphosate resistance, a neomycin phosphotransferase II (nptII) gene conferring resistance to kanamycin (Fraley et al., 1983), a hygromycin phosphotransferase gene conferring resistance to the antibiotic hygromycin (Vanden Elzen et al., 1985), genes conferring resistance to glufosinate or broxynil (Comai et al., 1985; Gordon-Kamm et al., 1990; Stalker et al., 1988) such as dihydrofolate reductase and acetolactate synthase (Eichholtz et al., 1987, Shah et al., 1986, Charest et al., 1990). Further examples include mutant ALS and AHAS enzymes conferring resistance to imidazalinone or a sulfonylurea (Lee et al., 1988; Mild et al., 1990), a phosphinothricin-acetyl-transferase gene conferring phosphinothricin resistance (European Appln. 0 242 246), genes conferring resistance to phenoxy proprionic acids and cycloshexones, such as sethoxydim and haloxyfop (Marshall et al., 1992); and genes conferring resistance to triazine (psbA and gs+ genes) and benzonitrile (nitrilase gene) (Przibila et al., 1991).

A plant of the invention may also comprise a gene that confers resistance to insect, pest, viral or bacterial attack. For example, a gene conferring resistance to a pest, such as soybean cyst nematode was described in PCT Application WO96/30517 and PCT Application WO93/19181. Jones et al., (1994) describe cloning of the tomato Cf-9 gene for resistance to Cladosporium fulvum); Martin et al., (1993) describe a tomato Pto gene for resistance to Pseudomonas syringae pv. and Mindrinos et al., (1994) describe an Arabidopsis RSP2 gene for resistance to Pseudomonas syringae. Bacillus thuringiensis endotoxins may also be used for insect resistance. (See, for example, Geiser et al., (1986). A vitamin-binding protein such as avidin may also be used as a larvicide (PCT application US93/06487).

The use of use of viral coat proteins in transformed plant cells is known to impart resistance to viral infection and/or disease development affected by the virus from which the coat protein gene is derived, as well as by related viruses. (See Beachy et al., 1990). Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus. Id. Developmental-arrestive proteins produced in nature by a pathogen or a parasite may also be used. For example, Logemann et al., (1992), have shown that transgenic plants expressing the barley ribosome-inactivating gene have an increased resistance to fungal disease.

Transgenes may also be used conferring increased nutritional value or another value-added trait. One example is modified fatty acid metabolism, for example, by transforming a plant with an antisense gene of stearoyl-ACP desaturase to increase stearic acid content of the plant. (See Knutzon et al., 1992). A sense desaturase gene may also be introduced to alter fatty acid content. Phytate content may be modified by introduction of a phytase-encoding gene to enhance breakdown of phytate, adding more free phosphate to the transformed plant. Modified carbohydrate composition may also be affected, for example, by transforming plants with a gene coding for an enzyme that alters the branching pattern of starch. (See Shiroza et al., 1988) (nucleotide sequence of Streptococcus mutans fructosyltransferase gene); Steinmetz et al., (1985) (nucleotide sequence of Bacillus subtilis levansucrase gene); Pen et al., (1992) (production of transgenic plants that express Bacillus licheniformis α-amylase); Elliot et al., (1993) (nucleotide sequences of tomato invertase genes); Søgaard et al., (1993) (site-directed mutagenesis of barley α-amylase gene); and Fisher et al., (1993) (maize endosperm starch branching enzyme II)).

Transgenes may also be used to alter protein metabolism. For example, U.S. Pat. No. 5,545,545 describes lysine-insensitive maize dihydrodipicolinic acid synthase (DHPS), which is substantially resistant to concentrations of L-lysine which otherwise inhibit the activity of native DHPS. Similarly, EP 0640141 describes sequences encoding lysine-insensitive aspartokinase (AK) capable of causing a higher than normal production of threonine, as well as a subfragment encoding antisense lysine ketoglutarate reductase for increasing lysine.

In another embodiment, a transgene may be employed that alters plant carbohydrate metabolism. For example, fructokinase genes are known for use in metabolic engineering of fructokinase gene expression in transgenic plants and their fruit (see U.S. Pat. No. 6,031,154). A further example of transgenes that may be used are genes that alter grain yield. For example, U.S. Pat. No. 6,486,383 describes modification of starch content in plants with subunit proteins of adenosine diphosphoglucose pyrophosphorylase (“ADPG PPase”). In EP0797673, transgenic plants are discussed in which the introduction and expression of particular DNA molecules results in the formation of easily mobilized phosphate pools outside the vacuole and an enhanced biomass production and/or altered flowering behavior. Still further known are genes for altering plant maturity. U.S. Pat. No. 6,774,284 describes DNA encoding a plant lipase and methods of use thereof for controlling senescence in plants. U.S. Pat. No. 6,140,085 discusses FCA genes for altering flowering characteristics, particularly timing of flowering. U.S. Pat. No. 5,637,785 discusses genetically modified plants having modulated flower development such as having early floral meristem development and comprising a structural gene encoding the LEAFY protein in its genome.

Genes for altering plant morphological characteristics are also known and may be used in accordance with the invention. U.S. Pat. No. 6,184,440 discusses genetically engineered plants which display altered structure or morphology as a result of expressing a cell wall modulation transgene. Examples of cell wall modulation transgenes include a cellulose binding domain, a cellulose binding protein, or a cell wall modifying protein or enzyme such as endoxyloglucan transferase, xyloglucan endo-transglycosylase, an expansin, cellulose synthase, or a novel isolated endo-1,4-β-glucanase.

Methods for introduction of a transgene are well known in the art and include biological and physical, plant transformation protocols. See, for example, Miki et al. (1993).

Once a transgene is introduced into a variety it may readily be transferred by crossing. By using backcrossing, essentially all of the desired morphological and physiological characteristics of a variety are recovered in addition to the locus transferred into the variety via the backcrossing technique. Backcrossing methods can be used with the present invention to improve or introduce a characteristic into a plant (Poehlman et al., 1995; Fehr, 1987a,b).

### IV. TISSUE CULTURES AND IN VITRO REGENERATION OF SOYBEAN PLANTS

A further aspect of the invention relates to tissue cultures of a soybean variety of the invention. As used herein, the term “tissue culture” indicates a composition comprising isolated cells of the same or a different type or a collection of such cells organized into parts of a plant. Exemplary types of tissue cultures are protoplasts, calli and plant cells that are intact in plants or parts of plants, such as embryos, pollen, flowers, leaves, roots, root tips, anthers, and the like. In a preferred embodiment, the tissue culture comprises embryos, protoplasts, meristematic cells, pollen, leaves or anthers.

Exemplary procedures for preparing tissue cultures of regenerable soybean cells and regenerating soybean plants therefrom, are disclosed in U.S. Pat. No. 4,992,375; U.S. Pat. No. 5,015,580; U.S. Pat. No. 5,024,944, and U.S. Pat. No. 5,416,011, each of the disclosures of which is specifically incorporated herein by reference in its entirety.

An important ability of a tissue culture is the capability to regenerate fertile plants. This allows, for example, transformation of the tissue culture cells followed by regeneration of transgenic plants. For transformation to be efficient and successful, DNA must be introduced into cells that give rise to plants or germ-line tissue.

Soybeans typically are regenerated via two distinct processes; shoot morphogenesis and somatic embryogenesis (Finer, 1996). Shoot morphogenesis is the process of shoot meristem organization and development. Shoots grow out from a source tissue and are excised and rooted to obtain an intact plant. During somatic embryogenesis, an embryo (similar to the zygotic embryo), containing both shoot and root axes, is formed from somatic plant tissue. An intact plant rather than a rooted shoot results from the germination of the somatic embryo.

Shoot morphogenesis and somatic embryogenesis are different processes and the specific route of regeneration is primarily dependent on the explant source and media used for tissue culture manipulations. While the systems are different, both systems show variety-specific responses where some lines are more responsive to tissue culture manipulations than others. A line that is highly responsive in shoot morphogenesis may not generate many somatic embryos. Lines that produce large numbers of embryos during an ‘induction’ step may not give rise to rapidly-growing proliferative cultures. Therefore, it may be desired to optimize tissue culture conditions for each soybean line. These optimizations may readily be carried out by one of skill in the art of tissue culture through small-scale culture studies. In addition to line-specific responses, proliferative cultures can be observed with both shoot morphogenesis and somatic embryogenesis. Proliferation is beneficial for both systems, as it allows a single, transformed cell to multiply to the point that it will contribute to germ-line tissue.

Shoot morphogenesis was first reported by Wright et al. (1986) as a system whereby shoots were obtained de novo from cotyledonary nodes of soybean seedlings. The shoot meristems were formed subepidermally and morphogenic tissue could proliferate on a medium containing benzyl adenine (BA). This system can be used for transformation if the subepidermal, multicellular origin of the shoots is recognized and proliferative cultures are utilized. The idea is to target tissue that will give rise to new shoots and proliferate those cells within the meristematic tissue to lessen problems associated with chimerism. Formation of chimeras, resulting from transformation of only a single cell in a meristem, are problematic if the transformed cell is not adequately proliferated and does not give rise to germ-line tissue. Once the system is well understood and reproduced satisfactorily, it can be used as one target tissue for soybean transformation.

Somatic embryogenesis in soybean was first reported by Christianson et al. (1983) as a system in which embryogenic tissue was initially obtained from the zygotic embryo axis. These embryogenic cultures were proliferative but the repeatability of the system was low and the origin of the embryos was not reported. Later histological studies of a different proliferative embryogenic soybean culture showed that proliferative embryos were of apical or surface origin with a small number of cells contributing to embryo formation. The origin of primary embryos (the first embryos derived from the initial explant) is dependent on the explant tissue and the auxin levels in the induction medium (Hartweck et al., 1988). With proliferative embryonic cultures, single cells or small groups of surface cells of the ‘older’ somatic embryos form the ‘newer’ embryos.

Embryogenic cultures can also be used successfully for regeneration, including regeneration of transgenic plants, if the origin of the embryos is recognized and the biological limitations of proliferative embryogenic cultures are understood. Biological limitations include the difficulty in developing proliferative embryogenic cultures and reduced fertility problems (culture-induced variation) associated with plants regenerated from long-term proliferative embryogenic cultures. Some of these problems are accentuated in prolonged cultures. The use of more recently cultured cells may decrease or eliminate such problems.

### V. UTILIZATION OF SOYBEAN PLANTS

A soybean plant provided by the invention may be used for any purpose deemed of value. Common uses include the preparation of food for human consumption, feed for non-human animal consumption and industrial uses. As used herein, “industrial use” or “industrial usage” refers to non-food and non-feed uses for soybeans or soy-based products.

Soybeans are commonly processed into two primary products, soybean protein (meal) and crude soybean oil. Both of these products are commonly further refined for particular uses. Refined oil products can be broken down into glycerol, fatty acids and sterols. These can be for food, feed or industrial usage. Edible food product use examples include coffee creamers, margarine, mayonnaise, pharmaceuticals, salad dressings, shortenings, bakery products, and chocolate coatings.

Soy protein products (e.g., meal), can be divided into soy flour concentrates and isolates which have both food/feed and industrial use. Soy flour and grits are often used in the manufacturing of meat extenders and analogs, pet foods, baking ingredients and other food products. Food products made from soy flour and isolate include baby food, candy products, cereals, food drinks, noodles, yeast, beer, ale, etc. Soybean meal in particular is commonly used as a source of protein in livestock feeding, primarily swine and poultry. Feed uses thus include, but are not limited to, aquaculture feeds, bee feeds, calf feed replacers, fish feed, livestock feeds, poultry feeds and pet feeds, etc.

Whole soybean products can also be used as food or feed. Common food usage includes products such as the seed, bean sprouts, baked soybean, full fat soy flour used in various products of baking, roasted soybean used as confectioneries, soy nut butter, soy coffee, and other soy derivatives of oriental foods. For feed usage, hulls are commonly removed from the soybean and used as feed.

Soybeans additionally have many industrial uses. One common industrial usage for soybeans is the preparation of binders that can be used to manufacture composites. For example, wood composites may be produced using modified soy protein, a mixture of hydrolyzed soy protein and PF resins, soy flour containing powder resins, and soy protein containing foamed glues. Soy-based binders have been used to manufacture common wood products such as plywood for over 70 years. Although the introduction of urea-formaldehyde and phenol-formaldehyde resins has decreased the usage of soy-based adhesives in wood products, environmental concerns and consumer preferences for adhesives made from a renewable feedstock have caused a resurgence of interest in developing new soy-based products for the wood composite industry.

Preparation of adhesives represents another common industrial usage for soybeans. Examples of soy adhesives include soy hydrolyzate adhesives and soy flour adhesives. Soy hydrolyzate is a colorless, aqueous solution made by reacting soy protein isolate in a 5 percent sodium hydroxide solution under heat (120° C.) and pressure (30 psig). The resulting degraded soy protein solution is basic (pH 11) and flowable (approximately 500 cps) at room temperature. Soy flour is a finely ground, defatted meal made from soybeans. Various adhesive formulations can be made from soy flour, with the first step commonly requiring dissolving the flour in a sodium hydroxide solution. The strength and other properties of the resulting formulation will vary depending on the additives in the formulation. Soy flour adhesives may also potentially be combined with other commercially available resins.

Soybean oil may find application in a number of industrial uses. Soybean oil is the most readily available and one of the lowest-cost vegetable oils in the world. Common industrial uses for soybean oil include use as components of anti-static agents, caulking compounds, disinfectants, fungicides, inks, paints, protective coatings, wallboard, anti-foam agents, alcohol, margarine, paint, ink, rubber, shortening, cosmetics, etc. Soybean oils have also for many years been a major ingredient in alkyd resins, which are dissolved in carrier solvents to make oil-based paints. The basic chemistry for converting vegetable oils into an alkyd resin under heat and pressure is well understood to those of skill in the art.

Soybean oil in its commercially available unrefined or refined, edible-grade state, is a fairly stable and slow-drying oil. Soybean oil can also be modified to enhance its reactivity under ambient conditions or, with the input of energy in various forms, to cause the oil to copolymerize or cure to a dry film. Some of these forms of modification have included epoxidation, alcoholysis or tranesterification, direct esterification, metathesis, isomerization, monomer modification, and various forms of polymerization, including heat bodying. The reactive linoleic-acid component of soybean oil with its double bonds may be more useful than the predominant oleic- and linoleic-acid components for many industrial uses.

Solvents can also be prepared using soy-based ingredients. For example, methyl soyate, a soybean-oil based methyl ester, is gaining market acceptance as an excellent solvent replacement alternative in applications such as parts cleaning and degreasing, paint and ink removal, and oil spill remediation. It is also being marketed in numerous formulated consumer products including hand cleaners, car waxes and graffiti removers. Methyl soyate is produced by the transesterification of soybean oil with methanol. It is commercially available from numerous manufacturers and suppliers. As a solvent, methyl soyate has important environmental- and safety-related properties that make it attractive for industrial applications. It is lower in toxicity than most other solvents, is readily biodegradable, and has a very high flash point and a low level of volatile organic compounds (VOCs). The compatibility of methyl soyate is excellent with metals, plastics, most elastomers and other organic solvents. Current uses of methyl soyate include cleaners, paint strippers, oil spill cleanup and bioremediation, pesticide adjuvants, corrosion preventives and biodiesel fuels additives.

### VI. KITS

Any of the compositions described herein may be comprised in a kit. In a non-limiting example, a composition for the detection of a polymorphism as described herein and/or additional agents, may be comprised in a kit. The kits may thus comprise, in suitable container means, a probe or primer for detection of the polymorphism and/or an additional agent of the present invention. In specific embodiments, the kit will allow detection of at least one allele associated with increased α′-subunit levels, for example, by detection of polymorphisms in such alleles and/or otherwise in linkage disequilibrium with the allele(s).

The kits may comprise a suitably aliquoted agent composition(s) of the present invention, whether labeled or unlabeled for any assay format desired to detect such alleles. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the detection composition and any other reagent containers in close confinement for commercial sale. Such containers may include injection or blow-molded plastic containers into which the desired vials are retained.

When the components of the kit are provided in one and/or more liquid solutions, the liquid solution may be an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means. The container means will generally include at least one vial, test tube, flask, bottle, syringe and/or other container means, into which the composition for detecting a null allele are placed, preferably, suitably allocated. The kits may also comprise a second container means for containing a sterile buffer and/or other diluent.

The kits of the present invention will also typically include a means for containing the vials in close confinement for commercial sale, such as, e.g., injection and/or blow-molded plastic containers into which the desired vials are retained. Irrespective of the number and/or type of containers, the kits of the invention may also comprise, and/or be packaged with, an instrument for assisting with the use of the detection compositions.

### VII. DEFINITIONS

In the description and tables which follow, a number of terms are used. In order to provide a clear and consistent understanding of the specification and claims, the following definitions are provided:

α-subunit: As used herein, means the β-conglycinin α-subunit.

α′-subunit: As used herein, means the β-conglycinin α′-subunit.

β-subunit: As used herein, means the β-conglycinin β-subunit.

A: When used in conjunction with the word “comprising” or other open language in the claims, the words “a” and “an” denote “one or more.”

Agronomically Elite: As used herein, means a genotype that has a culmination of many distinguishable traits such as seed yield, emergence, vigor, vegetative vigor, disease resistance, seed set, standability and threshability which allows a producer to harvest a product of commercial significance.

Allele: Any of one or more alternative forms of a gene locus, all of which alleles relate to a trait or characteristic. In a diploid cell or organism, the two alleles of a given gene occupy corresponding loci on a pair of homologous chromosomes.

Backcrossing: A process in which a breeder repeatedly crosses hybrid progeny, for example a first generation hybrid (F₁), back to one of the parents of the hybrid progeny. Backcrossing can be used to introduce one or more single locus conversions from one genetic background into another.

Consensus sequence: a constructed DNA sequence which identifies SNP and Indel polymorphisms in alleles at a locus. Consensus sequence can be based on either strand of DNA at the locus and states the nucleotide base of either one of each SNP in the locus and the nucleotide bases of all Indels in the locus. Thus, although a consensus sequence may not be a copy of an actual DNA sequence, a consensus sequence is useful for precisely designing primers and probes for actual polymorphisms in the locus.

Commercially Significant Yield: A yield of grain having commercial significance to the grower represented by an actual grain yield of at least 95% of the check lines AG2703 and DKB23-51 when grown under the same conditions.

Crossing: The mating of two parent plants.

Cross-pollination: Fertilization by the union of two gametes from different plants.

Down-regulatory mutation: For the purposes of this application a down regulatory mutation is defined as a mutation that reduces the expression levels of a protein from a given gene. Thus a down-regulatory mutation comprises null mutations.

F₁ Hybrid: The first generation progeny of the cross of two nonisogenic plants.

Genotype: The genetic constitution of a cell or organism, or a particular allele at a specified locus present in an organism.

Genotyping: Delineating the type of allele at a specified locus present in an organism. This is often accomplished by performing marker assays on DNA samples extracted from the organism.

Glycinin null: Mutant soybean plants with mutations conferring reduced glycinin content and increased β-conglycinin content. Plants with increased β-conglycinin contents may have non-transgenic null alleles for Gy1, Gy2, Gy3, and/or Gy4.

Immediately adjacent: describes a nucleic acid molecule that hybridizes to DNA containing a polymorphism, refers to a nucleic acid that hybridizes to DNA sequences that directly abut the polymorphic nucleotide base position. For example, a nucleic acid molecule that can be used in a single base extension assay is “immediately adjacent” to the polymorphism.

INDEL: Genetic mutations resulting from insertion or deletion of nucleotide sequence.

Industrial use: A non-food and non-feed use for a soybean plant. The term “soybean plant” includes plant parts and derivatives of a soybean plant.

Interrogation position: a physical position on a solid support that can be queried to obtain genotyping data for one or more predetermined genomic polymorphisms.

Haplotype: a chromosomal region within a haplotype window defined by at least one polymorphic molecular marker. The unique marker fingerprint combinations in each haplotype window define individual haplotypes for that window. Further, changes in a haplotype, brought about by recombination for example, may result in the modification of a haplotype so that it comprises only a portion of the original (parental) haplotype operably linked to the trait, for example, via physical linkage to a gene, QTL, or transgene. Any such change in a haplotype would be included in our definition of what constitutes a haplotype so long as the functional integrity of that genomic region is unchanged or improved.

Haplotype window: a chromosomal region that is established by statistical analyses known to those of skill in the art and is in linkage disequilibrium. Thus, identity by state between two inbred individuals (or two gametes) at one or more molecular marker loci located within this region is taken as evidence of identity-by-descent of the entire region. Each haplotype window includes at least one polymorphic molecular marker. Haplotype windows can be mapped along each chromosome in the genome. Haplotype windows are not fixed per se and, given the ever-increasing density of molecular markers, this invention anticipates the number and size of haplotype windows to evolve, with the number of windows increasing and their respective sizes decreasing, thus resulting in an ever-increasing degree confidence in ascertaining identity by descent based on the identity by state at the marker loci.

Linkage: A phenomenon wherein alleles on the same chromosome tend to segregate together more often than expected by chance if their transmission was independent.

Marker: A readily detectable phenotype, preferably inherited in codominant fashion (both alleles at a locus in a diploid heterozygote are readily detectable), with no environmental variance component, i.e., heritability of 1. In addition “marker” may referred to a polymorphic nucleic acid sequence or nucleic acid feature. A “polymorphism” is a variation among individuals in sequence, particularly in DNA sequence, or feature, such as a transcriptional profile or methylation pattern. Useful polymorphisms include single nucleotide polymorphisms (SNPs), insertions or deletions in DNA sequence (Indels), simple sequence repeats of DNA sequence (SSRs) a restriction fragment length polymorphism, a haplotype, and a tag SNP. A genetic marker, a gene, a DNA-derived sequence, a RNA-derived sequence, a promoter, a 5′ untranslated region of a gene, a 3′ untranslated region of a gene, microRNA, siRNA, a QTL, a satellite marker, a transgene, mRNA, ds mRNA, a transcriptional profile, and a methylation pattern may comprise polymorphisms. In a broader aspect, a “marker” can be a detectable characteristic that can be used to discriminate between heritable differences between organisms. Examples of such characteristics may include genetic markers, protein composition, protein levels, oil composition, oil levels, carbohydrate composition, carbohydrate levels, fatty acid composition, fatty acid levels, amino acid composition, amino acid levels, biopolymers, pharmaceuticals, starch composition, starch levels, fermentable starch, fermentation yield, fermentation efficiency, energy yield, secondary compounds, metabolites, morphological characteristics, and agronomic characteristics.

Marker assay: a method for detecting a polymorphism at a particular locus using a particular method, e.g. measurement of at least one phenotype (such as seed color, flower color, or other visually detectable trait), restriction fragment length polymorphism (RFLP), single base extension, electrophoresis, sequence alignment, allelic specific oligonucleotide hybridization (ASO), random amplified polymorphic DNA (RAPD), microarray-based technologies, and nucleic acid sequencing technologies, single nucleotide polymorphism, etc.

Non-transgenic mutation: A mutation that is naturally occurring, or induced by conventional methods (e.g. exposure of plants to radiation or mutagenic compounds), not including mutations made using recombinant DNA techniques.

Null phenotype: A null phenotype as used herein means that a given protein is not expressed at levels that can be detected. In the case of the Gy subunits, expression levels are determined by SDS-PAGE and Coomassie staining.

Phenotype: The detectable characteristics of a cell or organism, which characteristics are the manifestation of gene expression.

Polymorphism: the presence of one or more variations of a nucleic acid sequence at one or more loci in a population of one or more individuals. The variation may comprise but is not limited to one or more base changes, the insertion of one or more nucleotides or the deletion of one or more nucleotides. A polymorphism includes a single nucleotide polymorphism (SNP), a simple sequence repeat (SSR) and indels, which are insertions and deletions. A polymorphism may arise from random processes in nucleic acid replication, through mutagenesis, as a result of mobile genomic elements, from copy number variation and during the process of meiosis, such as unequal crossing over, genome duplication and chromosome breaks and fusions. The variation can be commonly found or may exist at low frequency within a population, the former having greater utility in general plant breeding and the later may be associated with rare but important phenotypic variation.

Quantitative Trait Loci (QTL): Quantitative trait loci (QTL) refer to genetic loci that control to some degree numerically representable traits that are usually continuously distributed.

SNP: Refers to single nucleotide polymorphisms, or single nucleotide mutations when comparing two homologous sequences.

Soybean: Glycine max and includes all plant varieties that can be bred with soybean, including wild soybean species.

Stringent Conditions: Refers to nucleic acid hybridization conditions of 5×SSC, 50% formamide and 42° C.

Substantially Equivalent: A characteristic that, when compared, does not show a statistically significant difference (e.g., p=0.05) from the mean.

Tissue Culture: A composition comprising isolated cells of the same or a different type or a collection of such cells organized into parts of a plant.

Transgene: A genetic locus comprising a sequence which has been introduced into the genome of a soybean plant by transformation.

Typing: any method whereby the specific allelic form of a given soybean genomic polymorphism is determined. For example, a single nucleotide polymorphism (SNP) is typed by determining which nucleotide is present (i.e. an A, G, T, or C). Insertion/deletions (Indels) are determined by determining if the Indel is present. Indels can be typed by a variety of assays including, but not limited to, marker assays.

Nutraceutical: Foods that have a medicinal effect on human health.

### IX. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Genomic Region Associated with Increased α′-Subunit Phenotype

The relative percentages of α′, α, and β subunits in the β-conglycinin trimer are ˜35, 45, and 20%, respectively (Maruyama et al., 1999). The ratio of α:α′ is approximately 1.28 in most seeds. Select varieties were screened for increased α′-subunit content. Protein analysis was carried out as follows: soybean seeds from a single variety were pooled and ground using the CAT Mega-Grinder (SOP Asci-01-0002). Ground samples were stored at 4° C. For analysis, ˜30 mg of flour from each was weighed into one well of a 96 well 2 ml microtiter plate. Protein was extracted for 1 hour with shaking in 1.0 ml 1× Laemmli SDS buffer pH 6.8 containing 0.1M dithiothreitol (DTT) as a reductant. Following centrifugation, a portion of each extract was further diluted in SDS buffer to yield 0.2-0.5 μg/μL total protein, heated to 90-100° C. for 10 min, and cooled. For each sample, 1-2 μg total protein was loaded using a 12 channel pipet onto a 26 lane 15% T gradient Tris/HCl Criterion gel. Molecular weight standards and a parental control were included in two of the lanes in each gel. The gels were electrophoresed until the tracking dye reached the bottom of the gel ˜1.2 hrs, then stained overnight in Colloidal Coomassie Blue G-250, destained in DI water, and imaged using the GS800 Calibrated Densitometer. Quantitation was performed using Bio-Rad Quantity One™ Software. The software was used to determine the relative quantity of each band in the sample lane. The percent acidic glycinin and percent β-conglycinin protein subunit bands are reported as the relative percent of the total protein in the lane. The sample identities and weights are tracked using Master LIMS™.

Most varieties did not have an increase in α′ (Table 1). In addition, most varieties had an average α:α′ ratio of approximately 1.28. Varieties with unique seed composition, i.e. wherein the ratio of α:α′ was less than 1, were identified and selected for analysis. In addition, varieties with normal α′ levels were selected for comparison evaluations.

TABLE 1
Protein analysis for phenotyping levels of Glycinin, β-conglycinin
and β-conglycinin subunits

| | | Relative Percent of Protein | | | | |
|---|---|---|---|---|---|---|
| Variety | α:α′ | α′ βC | α βC | β βC | Total βC | Total Gly |
| MV0061 | 0.7 | 10.7 | 7.3 | 5.9 | 23.9 | 31.5 |
| MV0065 | 0.7 | 10.3 | 7.4 | 8.8 | 26.5 | 32.8 |
| MV0062 | 0.8 | 9.7 | 7.6 | 7.7 | 24.9 | 31.5 |
| MV0063 | 0.8 | 9.5 | 7.2 | 7.8 | 24.5 | 31.1 |
| MV0069 | 0.8 | 9.4 | 8 | 8.8 | 26.2 | 32.7 |
| MV0060 | 0.9 | 10.5 | 9.5 | 7.2 | 27.2 | 29.2 |
| MV0066 | 0.9 | 8.5 | 7.5 | 7.6 | 23.6 | 32.8 |
| MV0030 | 1.3 | 8.3 | 10.4 | 4.7 | 23.4 | 30.7 |
| MV0053 | 1.3 | 8.9 | 11.3 | 6 | 26.2 | 29.5 |
| MV0054 | 1.3 | 9.6 | 11.5 | 6.6 | 27.8 | 31.4 |
| MV0055 | 1.3 | 8.8 | 11.2 | 4.8 | 24.8 | 29.6 |
| MV0056 | 1.3 | 8.5 | 10.6 | 5.3 | 24.5 | 31 |
| MV0057 | 1.3 | 8.8 | 10.5 | 5.3 | 24.6 | 31.8 |
| MV0058 | 1.3 | 9.1 | 11.4 | 6.4 | 26.9 | 28.9 |
| MV0064 | 1.3 | 9.4 | 11.2 | 5.8 | 26.4 | 31.4 |
| MV0071 | 1.3 | 8.5 | 10.8 | 5.4 | 24.6 | 28.1 |
| MV0059 | 1.4 | 8.7 | 12.5 | 6 | 27.3 | 27 |
| MV0067 | 1.4 | 9.7 | 13.7 | 6.2 | 29.5 | 30.4 |
| MV0068 | 1.4 | 9.7 | 13.5 | 5.3 | 28.5 | 31 |
| MV0070 | 1.4 | 8.9 | 11.9 | 5.3 | 26.2 | 28.2 |

Soybean varieties with increased and normal α′ levels were fingerprinted with 1423 SNP markers and compared for polymorphic regions. The associations between SNP marker genotype and decrease α-subunit content resulting increased α′-subunit phenotype were evaluated. A region on LG I between 45-60.3 cM demonstrated polymorphisms between increased and decreased a levels lines and is reported in Table 2. The informative sequences for decreased a levels are listed in Table 3.

TABLE 2
Genotype of region associated with increased α′-subunit phenotype

| | | | | | | | | | Normal | |
|---|---|---|---|---|---|---|---|---|---|---|
| | | | Increased α′-subunit phenotype | | | | | | α′-subunit phenotype | |
| SEQ ID | Chromosome | Position (cM) | MV0061 | MV0064 | FAYETTE | INA | PI88788 | MV0111 | MV0103 | MV0109 |
| 1 | I | 45 | TT | TT | TT | TT | TT | TT | AA | AA |
| 2 | I | 47.9 | CC | CC | ** | CC | CC | CC | TT | TT |
| 3 | I | 48.7 | AA | AA | AA | AA | AA | AA | GG | GG |
| 4 | I | 48.7 | GG | GG | GG | GG | GG | GG | AA | AA |
| 5 | I | 48.7 | CC | CC | CC | CC | CC | CC | TT | TT |
| 6 | I | 48.7 | CC | CC | ** | CC | ** | CC | TT | TT |
| 7 | I | 49.1 | GG | GG | GG | GG | GG | GG | AA | AA |
| 8 | I | 49.4 | TT | ** | ** | TT | TT | ** | CC | CC |
| 9 | I | 51.6 | TT | ** | TT | ** | TT | TT | CC | CC |
| 10 | I | 53.1 | II | ** | ** | II | II | DI | DD | DD |
| 11 | I | 53.8 | CC | CC | CC | CC | CC | CC | TT | TT |
| 12 | I | 53.8 | AA | AA | ** | AA | AA | AA | GG | GG |
| 13 | I | 55.5 | CC | CC | ** | CC | CC | CC | CC | CC |
| 14 | I | 55.5 | AA | AA | ** | AA | ** | AA | GG | GG |
| 15 | I | 55.9 | CC | CC | ** | CC | CC | CC | AA | AA |
| 16 | I | 55.9 | CC | ** | ** | CC | CC | CC | CC | CC |
| 17 | I | 60.3 | TT | GT | TT | TT | TT | TT | GG | GG |
| 18 | I | 60.3 | GG | CG | ** | GG | GG | GG | CC | CC |

| | Normal α′-subunit phenotype | | | | | | | |
|---|---|---|---|---|---|---|---|---|
| SEQ ID | MV0059 | MV0030 | MV0110 | MV0040 | MV0046 | ESSEX | WILLIAMS | [symbol] 2 |
| 1 | AA | AA | AA | AA | AA | AA | AA | AA |
| 2 | TT | TT | TT | TT | TT | TT | TT | TT |
| 3 | GG | GG | GG | GG | GG | GG | GG | GG |
| 4 | AA | AA | AA | AA | AA | AA | AA | AA |
| 5 | TT | TT | TT | TT | TT | TT | TT | TT |
| 6 | ** | TT | TT | TT | TT | TT | TT | TT |
| 7 | AA | AA | AA | AA | AA | AA | AA | AA |
| 8 | CC | CC | CC | CC | CC | CC | CC | CC |
| 9 | ** | CC | CC | CC | CC | CC | CC | CC |
| 10 | DD | DD | DD | DD | DD | DD | DD | DD |
| 11 | TT | TT | TT | TT | TT | TT | TT | TT |
| 12 | GG | GG | GG | GG | GG | GG | GG | GG |
| 13 | CC | CC | CC | CC | CC | CC | CC | CC |
| 14 | ** | ** | GG | GG | GG | GG | GG | GG |
| 15 | AA | AA | AA | AA | AA | AA | AA | AA |
| 16 | CC | CC | CC | CC | CC | CC | CC | CC |
| 17 | GG | GG | GG | GG | GG | GG | GG | GG |
| 18 | ** | CC | CC | CC | CC | CC | CC | CC |

[symbol]

indicates data missing or illegible when filed

TABLE 3
Listing of SNP markers for reduce a-subunit with the alleles for
each marker indicated, where “D” designates a deletion
and “I” designates an insertion.

| | | | | Normal | | | | |
|---|---|---|---|---|---|---|---|---|
| | | | Decreased | α-- | | | | |
| SEQ | | Po- | α--subunit | subunit | Forward | Reverse | Probe | Probe |
| ID | LG | sition | allele | allele | Primer | Primer | 1 | 2 |
| 1 | I | 45 | TT | AA | 19 | 20 | 55 | 56 |
| 2 | I | 47.9 | CC | TT | 21 | 22 | 57 | 58 |
| 3 | I | 48.7 | AA | GG | 23 | 24 | 59 | 60 |
| 4 | I | 48.7 | GG | AA | 25 | 26 | 61 | 62 |
| 5 | I | 48.7 | CC | TT | 27 | 28 | 63 | 64 |
| 6 | I | 48.7 | CC | TT | 29 | 30 | 65 | 66 |
| 7 | I | 49.1 | GG | AA | 31 | 32 | 67 | 68 |
| 8 | I | 49.4 | TT | CC | 33 | 34 | 69 | 70 |
| 9 | I | 51.6 | TT | CC | 35 | 36 | 71 | 72 |
| 10 | I | 53.1 | II | DD | 37 | 38 | 73 | 74 |
| 11 | I | 53.8 | CC | TT | 39 | 40 | 75 | 76 |
| 12 | I | 53.8 | AA | GG | 41 | 42 | 77 | 78 |
| 13 | I | 55.5 | CC | CC | 43 | 44 | 79 | 80 |
| 14 | I | 55.5 | AA | GG | 45 | 46 | 81 | 82 |
| 15 | I | 55.9 | CC | AA | 47 | 48 | 83 | 84 |
| 16 | I | 55.9 | CC | CC | 49 | 50 | 85 | 86 |
| 17 | I | 60.3 | TT | GG | 51 | 52 | 87 | 88 |
| 18 | I | 60.3 | GG | CC | 53 | 54 | 89 | 90 |

### Example 2

Utility of Genetic Markers Associated with Increased α′-Subunit Across Different Genetic Backgrounds

Four populations were generated to verify alleles associated with increased α′-subunit content in seed of soybean. A decreased α-subunit line, MV0064 was crossed with two normal α-subunit line, MV0040 or MV0112, to create two populations. MV0064 has the decrease α-subunit content resulting increased α′-subunit content and shares the same common source of decreased α-subunit as MV0060 at the grandparent level. MV0040 or MV0112 share some common parents to MV0060, but have normal α-subunit content. The F₂ populations are phenotyped for α′-subunit and α-subunit content and screened with SNP markers identified in Example 1. Moreover, a population was developed by crossing MV0064 with low glycinin parent, MV0113. MV0113 has reduced glycinin content (5% of total protein) and increased beta-conglycinin content (48% of total protein). The low glycinin parent has mutant Gy alleles that reduce the level of glycininin and subsequently increase the level β-conglycinin in seed. The F₂ populations are phenotyped for α′-subunit and α-subunit content and screened with SNP markers identified in Example 1. The populations confirm the prediction ability of markers in the presence of mutant Gy alleles.

Hybrid seeds were harvested from each cross and replanted. The F₁ plants were confirmed to be true hybrids through phenotypic and/or molecular characterization. The increased α′-subunit phenotype was evaluated as described in Example 1. The F₂ seed from the F₁ plants of each of the three crosses was harvested and replanted. A tissue sample was taken from each individual F₂ plant in each population and the DNA was analyzed with SNP markers: SEQ ID NO: 11 and SEQ ID NO: 15. Association analysis has shown that increased α′-subunit varieties have CC nucleotides at both SEQ ID NO: 11 and SEQ ID NO: 15, while normal α′-subunit varieties have a TT and AA at SEQ ID NO: 11 and SEQ ID NO: 15, respectively.

The F₂ plants which were scored as CC at SEQ ID NO: 11 and SEQ ID NO: 15 were considered positive for the putative mutant allele, while plants which were scored as TT and AA at SEQ ID NO: 11 and SEQ ID NO: 15, respectively, were considered negative for the mutant allele. A single pod was harvested from each of the positive and each of the negative plants in each population and was used to form separate positive and negative single pod descent populations for each cross. The remaining F₃ seed from each positive and each negative F₂ plant from each population was threshed in bulk to form separate positive and negative bulk populations for each cross. Some of the bulk seed was used for to evaluate protein composition as described in Example 1.

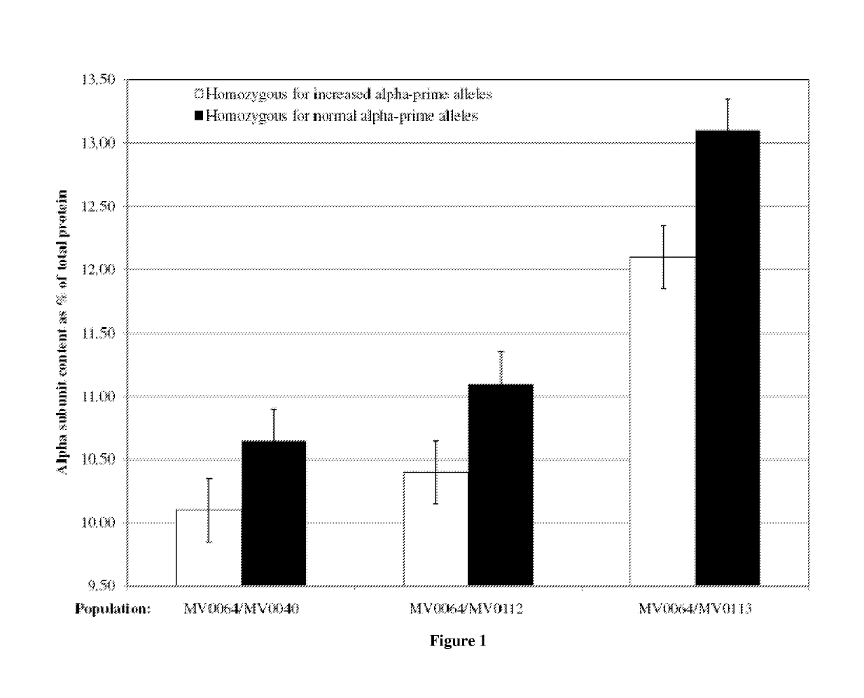

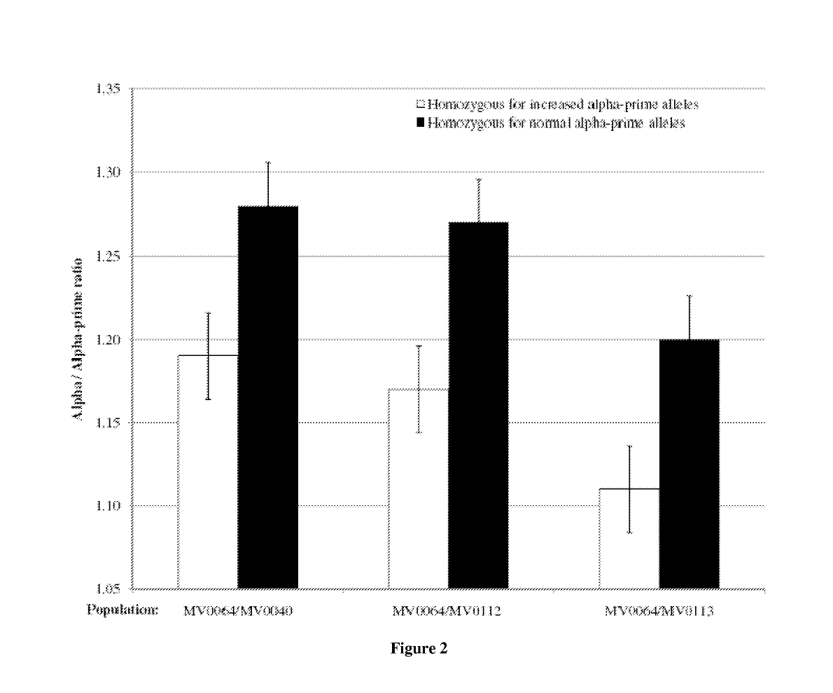

In three populations the presence of the putative mutant alleles (positive F₃ bulk) at both marker loci was associated with a 6.5% increase in α′-subunit content (p=0.015) (FIG. 1) and a 8% decrease in the α-subunit/α′-subunit ratio (p=0.0002) (FIG. 2). The markers were associated with 68% of the variation in α′-subunit content in the seed. There was no significant difference between positive and negative classes in the level of: α′-subunit (p=0.5), β-subunit (p=0.9) or total β-conglycinin (p=0.2). The screening of the three populations confirms that the marker is informative across different genetic backgrounds. Furthermore, the F₂ bulks derived from crosses between MV0064 with MV0040 or MV0112 were categorized into two classes at SEQ ID NO:11 and SEQ ID NO: 15, respectively: CCCC and TTAA. No plants with the TTCC or CCAA haplotype were observed. A sample from each F₂ bulk was planted. The plants were tissue sampled for genotyping with SEQ ID NO: 11 and SEQ ID NO:15. The F₃ seed of each plant was harvested individually (Table 4 and 5). Eight F₃ seed from each plant were used to evaluate for the α-subunit and α′-subunit contents using SDS-PAGE (Table 4 and 5).

The molecular markers, SEQ ID:11 and SEQ ID: 15, are useful in breeding for increase α′-subunit content in soybean. The phenotypic selection criteria for increased α′-subunit content is an α-subunit/α′-subunit greater than 1. Although the molecular markers are not entirely predictive for an α-subunit/α′-subunit ratio less than 1, the markers serve to reduce the population size required for phenotyping using SDS-PAGE. The cost of evaluating a single plant via SDS-PAGE for α-subunit and α′-subunit level is estimated at $18 a sample. In addition, genotyping plants followed by confirming the phenotype by SDS-PAGE reduces the expensive phenotyping cost by at least 50% (Table 4 and 5). In addition, the probability of obtaining a plant that meets the selection criteria of α-subunit/α′-subunit ratio less than 1 is greatly increased (Table 4 and 5).

TABLE 4
Utilizing molecular markers for selection of increase α′-subunit
levels within a population
Cross: MV0112/MV0064

| | SEQ ID NO: | SEQ ID NO: | SEQ ID NO: | SEQ ID NO: |
|---|---|---|---|---|
| | 11 | 15 | 11 | 15 |
| Allele | TT | AA | CC | CC |

| alpha/alpha- | Number | | Total |
|---|---|---|---|
| prime | of plants | Number of plants | Plants |
| >1.0 | 86 | 59 | 145 |
| <1.0 | 6 | 29 | 35 |
| Total Plants | 92 | 88 | 180 |
| % of plants meeting | 33% | % of plants meeting selection | 19% |
| selection criteria | | criteria without use of markers | |
| with use of markers | | | |
| Phenotyping costs | $1,584 | Phenotyping costs without use | $3,240 |
| with use of markers: | | of markers: | |

TABLE 5
Utilizing molecular markers for selection of increase α′-subunit
levels within a population
Cross: MV0040/MV0064

| | SEQ ID NO: | SEQ ID NO: | SEQ ID NO: | |
|---|---|---|---|---|
| | 11 | 15 | 11 | SEQ ID NO: 15 |
| Allele | TT | AA | CC | CC |

| alpha/alpha- | Number | | Total |
|---|---|---|---|
| prime | of plants | Number of plants | Plants |
| >1.0 | 107 | 84 | 191 |
| <1.0 | 5 | 23 | 28 |
| Total Plants | 112 | 107 | 219 |
| % of plants meeting | 21% | % of plants meeting selection | 13% |
| selection criteria | | criteria without use of markers | |
| with use of markers | | | |
| Phenotyping costs | $1,926 | Phenotyping costs without use | $3,942 |
| with use of markers: | | of markers: | |

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A soybean seed comprising a β-conglycinin trimer content wherein the subunit ratio of α:α′ is between about 0.1 and about 1, wherein said seed is produced by a method comprising the steps of:
A) genotyping a plurality of soybean plants with respect to a genetic locus on LG I;
B) selecting a soybean plant with a desirable genotype in said genetic locus that conditions a seed protein phenotype wherein the subunit ratio of α:α′ is between about 0.1 and about 1 in the β-conglycinin trimer; and
C) growing said selected plant to produce seeds, wherein at least one of the seeds produced has a seed protein phenotype wherein the subunit ratio of α:α′ is between about 0.1 and about 1 in the β-conglycinin trimer.

2. The soybean seed of claim 1, wherein said desirable genotype is selected from the group consisting of the genotypes of soybean varieties Fayette, Ina, PI88788, and progeny of these varieties having the desirable genotype.

3. The soybean seed of claim 1, wherein said desirable genotype is selected from the group consisting of the genotypes provided in Table 2 for soybean varieties MV0061, MV0064, and MV0111, when said desirable genotype is determined using one or more of the markers listed in Table 2.

4. The soybean seed of claim 1, wherein said subunit ratio of α:α′ is determined using SDS-PAGE.

5. The soybean seed of claim 1, wherein said subunit ratio of α:α′ is between about 0.2 and about 0.8.

6. The soybean seed of claim 1, wherein said subunit ratio of α:α′ is between about 0.4 and about 0.6.

7. A method for producing a soybean plant capable of producing seed comprising a β-conglycinin trimer content wherein the subunit ratio of α:α′ is between about 0.1 and about 1, comprising the steps of:
A) crossing at least one plant with decreased α-subunit resulting in increased α′-subunit levels in the β-conglycinin trimer with at least one plant with normal α′-subunit levels in order to form a segregating population;
B) genotyping at least one plant from said segregating population with respect to a genetic locus on LG I; and
C) selecting a soybean plant with a desirable genotype in said genetic locus that conditions a seed protein phenotype wherein the subunit ratio of α:α′ is between about 0.1 and about 1 in the β-conglycinin trimer.

8. The method of claim 7, wherein said desirable genotype is selected from the group consisting of the genotypes of soybean varieties Fayette, Ina, PI88788, and progeny of these varieties having the desirable genotype.

9. The method of claim 7, wherein said desirable genotype is selected from the group consisting of the genotypes provided in Table 2 for soybean varieties MV0061, MV0064, and MV0111, when said desirable genotype is determined using one or more of the markers listed in Table 2.

10. The method of claim 7, wherein said subunit ratio of α:α′ is determined using SDS-PAGE.

11. The method of claim 7, wherein said subunit ratio of α:α′ is between about 0.2 and about 0.8.

12. The method of claim 7, wherein said subunit ratio of α:α′ is between about 0.4 and about 0.6.

13. A method for selecting a soybean plant capable of producing seed comprising a β-conglycinin trimer content wherein the subunit ratio of α:α′ is between about 0.1 and about 1, comprising the steps of:
A) genotyping a plurality of soybean plants with respect to a genetic locus on LG I; and
B) selecting a soybean plant with a desirable genotype in said genetic locus that conditions a seed protein phenotype wherein the subunit ratio of α:α′ is between about 0.1 and about 1 in the β-conglycinin trimer.

14. The method of claim 13 further comprising the steps of allowing the selected soybean plant to set seed and screening the resulting seeds for β-conglycinin trimer subunit composition.

15. The method of claim 14 further comprising selecting from said resulting seeds a seed having a β-conglycinin trimer content wherein the subunit ratio of α:α′ is between about 0.1 and about 1.

16. The method of claim 15, wherein said subunit ratio of α:α′ is between about 0.2 and about 0.8.

17. The method of claim 16, wherein said subunit ratio of α:α′ is between about 0.4 and about 0.6.

18. The method of claim 13, wherein said desirable genotype is selected from the group consisting of the genotypes of soybean varieties Fayette, INA, PI88788, and progeny of these varieties having the desirable genotype.

19. The method of claim 13, wherein said desirable genotype is selected from the group consisting of the genotypes provided in Table 2 for soybean varieties MV0061, MV0064, and MV0111, when said desirable genotype is determined using one or more of the markers listed in Table 2.


SEQUENCE LISTING (90 sequences)

SEQ ID NO: 1 - DNA, 874 bp, Glycine max
agcctcactcctggtgatggccctaaatccaatttgtctaccaaatatggactcattttggcacctaaaacaacactcaaccgacagatacaaacctgaagagccagtatcctatcttctactgtatcttttatagtgattcgtgtcacagtaacaggacgagtttgcccaattctgtgagctcgatcaatagcttgatcttcagttgtcggattccaccaaagatccaaaagaataacatgacatgcagcaaccatattcaaaccaaggtttcctgcttttagtgacatcagcataacagttatctgaaacaaatttcaattagtcagacaccaaagtctaccaaggttaagacactagaacacagtagaaaattcaagttcaaggagttggaaaacatactgattaaatatgcaacaagcattattgatataaccattaagaggattaaatttcatactacttcatctactaagcaataggttcacatattacttggtgtcattaatagatttgcataaaaaagttacagctttaacaatgttgggaaaggccaaacctcaggttcagtattgaaatccttaacagctttgtccctcgcacccagagtcattctaccatcaagtctccggtactgtataccaaattgtttcaacgatgtctcaactaagtccagcatgctagtccactgggaaaaaactatagcctttattggtccttcagttgtggactctgaataccttcttgtgtgttttgtaaccctaacatctgaatcgcagtcttcaacatgtaaattatccaaagaaggtgaatctctacaacctccagaggagttcggtaagtcagaactagaaatcttcaatttacaatttga

SEQ ID NO: 2 - DNA, 593 bp, Glycine max
tttgcatgcctgcagccagtagggaaattgctctgatttataagttttgaattgttttctctttcacctttttcctctcctatttactattgcttgatttttaaattcattaaacaaaattgactagttgaaagatgacaattttatggcacaattagtttttttcttttcgaacctgtgagtagggatttatgcgatcaacagaagttctattaaggttcaaggggaagtttatctcataataagtgcaagtcacttttaatttttgtaggtccttccaaaagataaaggggaagttagcagatgaagtcatctttttttttttttaaaggttatttgttttagtaagctttgatatattggggtgacaaattttgtgattggggtgaagataggatgtatctgtcgaatgagaaattctgatttcgttcttgtgttgtactatctggcaggacgacaagagaaaaatggttgcatctgcttttggggaagaccatgctggcgcgtctggaactcgccttacagtagatgatctgaagtatcttttcatggtgtagactctagaccactccttgcttggtcttcccatgatg

SEQ ID NO: 3 - DNA, 648 bp, Glycine max
aaaaacgattcttgtccataacagactttattatgtaagcttccattccgtactcttcacaaagcttaactaactccaaggcatcaccagctgctttatacccaaattggtagttctcacctaccagaaagttccagaatccagtacagacttctatttcagcttatgtgtgagtaaatattggtaacataatatatcatgttgatgttgctattcaggaaaaataacaaaatacgagtaaattatgttaatattaaagggggtggtataccttgatggtaaattgtggagctacttatttacctgtggattgagatgccgcacacttgaaaactcaacctgaaactcttctgggaccatgttacaacaataagaaacccaagaagaaagaactcgcttttggtcacatttagcaactataggaggcctgtttgggaaacatttaacaaaatatacgttactttaccaatggagataacaattatgatgctctgataccccaacaattcagaacctaaacatgagttaggagtttatttatttactaaaattatgtcaagaactattttaacaaccataacatagccaaataatgtatctttcacgtatataataaccccaaagaactccaaaacaaagaccaaactt

SEQ ID NO: 4 - DNA, 886 bp, Glycine max
aactttacaaaaacaaaaacaaaggttgatcacaagattcctatcccgtcttccattatatatacaccagatcagatctatcatacgattttgagacaacttacaaattcccaataagacttctttcacaatatatttagttgcaattcttttttaaggtttccgggtcggatatacctttctaacaaaaattgaaaactcaaattgattctgctcagttcactttacttgtctaaatactacttttttttgggagaaaatagaatcatatctgatctttgaaaactcaaattgtttatcaagagaaaaacaagctggcaactaaacataaaaaattagtcctacatactaagaaaatttaatgtcgacatatctaatatattacccttttctcacatttttgttttgagtctgtcttgacttaaaagcctacttgaaggtctatttatgcaggcatgcttttaagtgagcaggttagcttgtgggctaacaggcaaagcctttttctaaataggttaaaagaacaattcctaaattaattgcacacgggagaaatatagaccatgacgagttcatatttcataaaggatgataatcaatattggatgtttggaagatgatctgtacaaaagtaacgagaagtttcttatcaacgctcaataacagcacctaaaatctcaatagttgggtgttttcatgcaatcccatgttccagctatctactacataaaaaagaaaggacctctgatcgaacaaagttccttgtcacgggaaataacatctcttacattcctcacaaggactattgtaaacatggcaatgctagcaatctattcacggaaactctgagctgcacaagatagtttccagttttacaacaacaaaa

SEQ ID NO: 5 - DNA, 983 bp, Glycine max
agctatacaaaacaaatataaaagccaatctcaagtttcttatcccgaccccttcttccatgatatacaccagaccaaacatttcataccattttggaacaacgggcttctccatgactacaaattcccccataagacttcattctcaataaatttagttacagggacatagcaattgacatctcaaattaattctgctcagtctgatttacttgttaaaatacaatttttatttggagaaattagaatcaagtttttttgtttttcaagggcaaaacaacctgacaactaaacataaaaaattagtcccgcatactatgtaaatttaatttcaacatatctagtatataaatttttcactattttgaactatgtatgcaaacaggtcaaaccaagccaatctttatggcttacatgtttaaaaaatataagacgtggtttagattatatgtcacgtttttgagtctgtcttgacttttactaaaaaggttaaaagaacaatttctaaatcaaaacgatttatacacgcaaaggagaactatagaccatgattagttcatattgcataaaaggatgataatcaatattgttggatgtttggaagatgatctgtacaaaaataacggcaaattcttatccacgctcagtacatcatagtacctaaaatctcaataatactgtgctttcatgcaatccctgtatcccatgttccatctatatatatatccactatatagaaaagaaaggccattgatcgaacaaagttcctggccacggaataatatctcttacaatcatcacactaacttttaaatagaaagatcattatttataaacagcacaaaacaaaaacacaataaggattaaaatgacaaataaattgttaataacacttctagtcatcatttgtttccagttgtctgtagactcatctctttgaacgttcccttccacatggcatgattttgaagaaaacaccactt

SEQ ID NO: 6 - DNA, 724 bp, Glycine max
aatacattctcctggtctcctttgctaatgtggacaacatccagagcttcctggaaattaaaaatagcatgaaaaaactcatgtatgtcaagctattatttcagactcaaaccacaagtcaaaatggaaattgtaagaaatcatatataaacttatgtgaatgcagatcattcataagaaaaaagtgatgaaaaataatgtataccttgactatgcgaaattcttctgcgtcatcaactcccgtaattgaataacaattgctctgcctcagatatttatagtcctcagcacttgttagatttagcttttctgcaatacaaagtcaaacatcaaacaacaatgaaaaggcaaacactaataaactagaatatacaacttaatgagttaatgtcatgtagaggagtattctagaataaaaagcttatatcattagggaaatcaaatcccatacatgtgcgattaggttctaattagtatcctcattagtttgttccaatcctgacccaactaaggagagatcaaagtcgaatgtctaattgagaaaaaggggagataaatgcccatttgactcagattgtaaaggaatgaatagtaatatgaaaagaagtaatgctaaaagagtttttttgagtattacaaagtgtgatagcattttttcaagttacaaggtttctccacattctattcagtgtgagcctaatcctcactcaaaatattgagcttt

SEQ ID NO: 7 - DNA, 818 bp, Glycine max
aatgggcatgacatgtcagcaacctgcacttgccttcgctgcaacttgtgtcttgtcggaagtctatccctaataagtctccaaacaaaaattgctattttgcttggaacctttatgcttcataatttgacaaaacagtcctcctgagttcctgctgctgctccttccatcagtaccttgtaagcactgtctgttgtgtaatgacctataggattagcagtccattcccacacatcaggtccatgatgtggaaaggtcatatcctaaatctcattgaggaagacacctactgaatcaatttcattatcaaaacatggccttctccaaataaaattccactcccacccgttgtctttgtagcttcccatctgttggatgaaactctgctgctgtaaggaaattgaacacagtctggggaatttttccgccagagacatctccccacatacccatttatcctcccaaaatttagtttgatccccacagcccaccttccaccgcataccactattaataatctaaccctgaggggaatgaatgagggccttcttcaagtctctccaccaaactgattctaaacctggtctatctgctgccaacataccctgccagccaccatacttagattgcactactctagcccaaagttctcctttgttttgcatcagaccccacctccatttaccaagcaatgctatgttgaaattggtgatatccttgatgtccagacctccattttccttggatgaagtcaccgtgtcccacctaatccatgcgatcttattttggtcaagccctcctccccaaagaaaccttcg

SEQ ID NO: 8 - DNA, 817 bp, Glycine max
gaaggtttctttggggaggagggcttgaccaaaataagatcgcatggattaggtgggacacggtgacttcatccaaggaaaatggaggtctggacatcaaggatatcaccaattttaacatagcattgcttggtaaatggaggtggggtctgatgcaaaacaaaggagaactttgggctagagtagtgcaatctaagtatggtggctgacagggtatgttggcagcagatagaccaggtttagaatcagtttggtggagagacttgaagaaggccctcattcattcccctcagggttagattattaatagtggtatgcggtggaaggtgggctgtggggatcaaactaaattttgggaggataagtgggtatgtggggagatgtctctggcggaaaaattccccagattgtgttcaatttccttacagcagcagagtttcatccaacagatgggaagctacaaagacaacgggtgggagtggaattttatttggagaaggccatgttttgataatgaaattgattcagtaggtgtcttcctcaatgagattcaggatatgacctttccacatcatggacctgatgtgtgggaatggactgctaatcctataggtcattacacaacagacagtgcttacaaggtactgatggaaggagcagcagcaggaactcaggaggactgttttgtcaaattatgaagcataaaggttccaaacaaaatagcaatttttgtttggagacttattagggatagacttccgacaagacacaagttgcagcgaaggcaagtgcaggttgctgacatgtcatgcccatt

SEQ ID NO: 9 - DNA, 793 bp, Glycine max
agaaaatatgtccactgttagattaaaaagaatgaaagatactaaaagctggggaaagtcatttagaataatttacaaagaattataatattcttaagtttaagttatagttctaatctaacatatttatatgattttctagattttaatattttctttttacaaaaagcatgccccctgcaaattttggctctagctctgccaccatgagcatagacaaaaaaataaaaatgaacaagggatttctcatcaatacaatgaaaattcagtgaagaaacctgataggattatggatctaattgggcccaataaggctctaggtttacttctttcagcctacacctaacttgcagggaactaacatgtatacataaaaataaaagggagaattagtgagagaagagagaaaacaaatctcagatctgtctaccttcaaagagggacagtgatcatgttagtattggcagtaggtaccaggtccacaggacctgtgagtgcaagccacacaagtatgacagaattgtttgtccagaatattgcctagttcccaaagccttgagttaattatggttgccctgaattgtttgtccaaatatgcacacattgcatacccttgaaacattttattaccaagactaataatgttgagatttttgttaaagtgatggtaagactacacaggtttcattgatctgtgatacatgatagaaatcctcctttcctaagtgatttttgggaagggctgatgatctgtttgaatacaagcagaaaaaaatgttaaaaataggtattt

SEQ ID NO: 10 - DNA, 564 bp, Glycine max
aaaaaataattatacttgactgatccatatcaagccaaccatcaaataagctcacaagaaaaatcaaccagcaacctcaaccagacataaaagtaatgcctgaatcacaagcaaaagtactcaagatcaacctgatactcagcaaattcaactgccagttccttgaacgctttgtctgctggttgaagtaacttatgggcttcctgaaattcgacaagaatgggatttcatggaagaatatgcaaaaactatcagcaccaagtagaacaaataaaacaatattagatgaatcatccacaacatattatgcagatgaatattttacatatttgctaatataaatcaaatgtcaaatattacatctatgaaagttggtatcctttccattttcatcactagatacaatgggacttgcaaatatttggaatgaatctcatcccatgtcaccatctatcaaggttgagcttataacaaggaaatgacataaataacaattgatatattttctattaaaaagaaaagaatcaacaattcaacaaccaaattgagacaaataccttttca

SEQ ID NO: 11 - DNA, 780 bp, Glycine max
agaacatttgctgctgcttctctcagtttatccatcttctccacagcttgcttacaaattcctccaactaaattggtagcaagattttcattgaataaaaaaagctctcggttgttcttaagcatgctatcaatcgaaggatatgcaataggttcaatttcatttccatctgatcttccagacaaacaaactgacttgtctatcttacagagcatgtatgtacatttttctaggccatccaatgcagcctcacgaacccaagaacctacatcacctctattatcaacagaataatcatcaagagctttaaataaacttatcatcacctcattctttatcagaataaacagggaaaaatcatcctcaacaaaagaggtagcagtatcttctcttccattaattaatgtttcacacactaatgtgagccctttgacagcatttactcgtgcttcagcatctctgtcttcagggttttcctgcacatgggaaaacattgtgtaacacaatcattgaacctaagattgtataatatatagcatttgcaatgtggagcacctcaattttacaagagccacaaagcttcaaaagcacatttctccattgactggctaataactcatatggcaaaacacctattgccaatgcagatcctctccttacagctacatttggatcagtcaacatactggaagtacctttgcttgtcaccatcactttattacctttattattcttgaaagccatggccaataattgccacccggattaaaagtggttttc

SEQ ID NO: 12 - DNA, 1240 bp, Glycine max
ttgcatgcctgcaggttaaattctatctgcacacttaagcacacacttatagaaccaagagcttgcatgaatgattctgaatggaaagcaaatgattccaatagcgtggctttggagaagttcaaaagagacagcgtttatattgacaaaaatggcagattaaggaacttcaatcacaaaaaagtgtcaaggaaaaaatgtaattaacacatcatggaacatgagtttcctcactagctcattgaaacgtgtgtgtcatgtttgaaaagtgtgttgttttacatatgggcaggtggttctttgagaggacgaggatggaaatacggttctgggtttgttgatgggatttttcctgtgctgagtccaactgcacagcagattctggactatgttgaaaagggtgtggagagtgagagcatttggggttctttggacatgcttcctcccactcttgatgcatgggatgatattttcactgtggctgttcaacttcggatgaggaaacagtgggattcaatcatttcggtgagaaactcatccctggttgcccttctaacttcttaacttcaaaaactaatttatctcttttcagaattattcaatggaatgcctaatgatttctctgaaaatctataacaatatataaagataatacggataccaattcttagtgtagacatttcttaacccattttatattcaactgatagtgatgtattgtaactccacactcaaattttagttttctttttgaagtagtgctaccttgtaaattaattattctcaaaaaaatgtggtgtctgtctttctgatagtgtttagtaatgatgatatttgtttcactcgcttctagcaaatttaccttactggtacagtttttatcagtcgaattatttgaagcgtgaaggttgacttatctcttgtaacgtttcaccattgaatccatcatgcattcatgttcatgtgtcacttgtacaccattgaatccatcatgcattcatgttcatgttcacttgggcaatattatgacactaattttttgtttttgtttattgcaattcttaatttattttgggattggctagtggatttgtgttgcccgtcataacagaaaaaaactgtacagatatgtagatggatactgctaaggagctcctttaagccagatgtaatctgctataatttactcatagaagcttttgggcaaaagcttctatacaaggaggctgaatccacatat

SEQ ID NO: 13 - DNA, 1070 bp, Glycine max
atttctgtcattaaaataggggttgacacatactgtaattcataaaggtgggtcgtgggagggtggtctgctaatgattattctattgtagtattgagctcatattatctgtgatttataccaaggggtgggaataaaggtgactggtaaatgatatgttgtgaaagtttttaggattcatgtggaggcttatttcagaataatggtggagagtgacatgctgtttataatttcagggaaaattgggaatcaagctaataatgtttgaataattttgactgtcaaaacgaagataaacttattagttgaaggctacagaaggggaatagaattttcactaaaccacaaaaaaaaaaaattgaaatatggatctcctggtttttgattttttttttcttgacatccatttgtttattaattcaattgattgttcatataaacttggatttttttctcttcaacttacatcgaaaggatttctctctctgcttctgaagtttgcaccaaaagaaaaaaacacaacattaaggatcttcaatattatgcattgccatttctggatgccacccatagatatagactatgtttttgtttagactgatgagtaggtttgatataggattacagttaattggacataattctgaagataagaatttaggaagggaaagtgttatttaaagtgttaagataccagtatataatttggccaacttctgttttgatgcactttgtgttactattggaagcattattatttatttgatacctctccctcctggttcaatgttgatagtgaagcattttacttacagtatttcccccatatgttatatctataatattcaatttattgaataaacaatgttaagaagatagaaaatgaatgatcagtagactaatctgtttgtgatgtgctgcaatttacctgattacaatatgttcttgtctggaagtatttgctggttttcttattgagttggacttactcaaagacaattttctattgtattcctgtaaatacttttaaaacagttttggttcatgaatatttaatacttagtatgccc

SEQ ID NO: 14 - DNA, 814 bp, Glycine max
aatagcaccagatgaactgcattcatagttacagttctgcaccagaaagctcttgctacagaggaagttgcatacatacatgacaacaaaactgtgattccatcccatgatttgtgccaaaccagcaccatttcccaaacataaacaggacaaaggagaaagcagtggtaataaggagaaacattctatctcaaaatcaattccgatctcacaaatgcacagccaaattcaaacagagagtacacaaaactaaccacaaataagagatttcattaaactaccatctcagaataagttccatatctagaatttaagattaaagtttaaagttcccacagtataacttcatccttgcagctacatacatagtggctacaaaagcaaagtagtaaactaaagcatgtgatttggaacaatcatcactgttgagagtattattctataaacaaaagaatgaaactatacttctgaaatttcaaaagcgacattgaaatcaacaaaggcacacgactgctcctgggaaaccccaccaagtccatccatccatcaaatcctcggatgtatcctaagcagccaacaacagaatgcctctccccaatctcaacgctacctctgtcaaaaaccaaagccaaccgttaattcacacgcaatccaaaacaaaacccaaaacttaatccttttttcaaacctcaaatgaacaaaattcacaaaaaagcagagtgtaacattcacataccttgccagtctcagcatcatccggaaccttgtaggaatcggcacaaacctccgacaaccactggccagggaaaagggt

SEQ ID NO: 15 - DNA, 1536 bp, Glycine max
ttcggactcgtacccgggcatctctaaatcgacctgcagtgcaaacaatgaaggttatctgttggaaaattcttcctgtttcatacatctgtttggatcatgtgaaaagtttgtgtggaactacataatgaagcactagtagcatcctgagatattctttggatatagtaattagaaatataataataagaaatgctagctacacactttcagaaatgctcttttcaagtcacactctttactattgggtgcattgttttgtgggtactgctccctttctagtgggtcatgcataaatttcacccaataacaaaaggtgtgttgctacttgctagccgttctcatacataatatatggccataaattatgatttcctcattcacacaacttgtgctacttatatttgatttcatgaacattttggattcgacacagtgcaacatgcaattaacaagtatctgtaattgcattttctttattgacagggtttgtttttaccttcagtcatttctctagttgttcctctggttctgatctccttgactaggtagagactcttcttcctacactgcaaaagtcagctgcaaaagctgatttgaatagtaagatttagcttaacatataatgttaggaacttggcaatttctctattgaagtatcctaaaaaatagaaagaaaagaggaaagatttgaaaatatgatgaaagtgttattactgaataggaggtacaataagccttccgaggacaatttagatgatgctagttctttacttttttcagtggagttaatgggaaggaacaaaaggtctctggatgtctttgcctctgaaccgattgctcctcgagggcaacttgttttctcagtgagtttaggagctttgatttttgtcccagtgttcaggtccctcacaggtttacctccgtacatcggaatgctgctcggacttggcatgctttggattttcgttgatgctatccattatggtgaatctgaaaggcagaagctaaaagtgccacatgctctgtcaaggatagacactcaaggagcactatttttcttgggaattctattatccgttagcaggtagtgcggaaatatattttaatttttatgctgtgataagttttggacaataaccatgtattaatgcattaaaaacaattataaaatacatcaagtcatcgacaaaagtgtcattgtccctttgagtagtagggcatttgctatgacttaataggtctgatatccacaaagtctaacattctggaaagatgatatattaccttgtttttacctttttcctatattatgagatgcatatattgttcttttgcatgaactgtgattacatattcttttgctgacatatctttaaataacctagttacttatgttagccggttgtatttgatcaattttaaccatcatgttcggcagcctggaggtagcagggattcttcgggaaatagcaaattactttgatgcacatgtcccaagatgtgaactgattgcaagtgctattggactaatat

SEQ ID NO: 16 - DNA, 1111 bp, Glycine max
aagtcagtgaaatagtgtttttcttgcttggtgcaatgaccattgttgagatagttgacactcatggaggatttaagctggttacagacaatataacaacccaaaacccacgccttctcctatgggtggtaagtgctttcactgctctgtcactgcattgttgttcaacattacagtctatggtcaaatttggatgaaacacacaagagaaggggctcagattcaagagctccttcttacaaaggggtctagtactatttatttaggctgtgcatccatgatagcaaacttttcaagagcaaattcttcctctccccctactcgaattctaaattttctgtagtttgcatcactattttttttgtctctaatggaaattgactaattaatgcttaatttgcagattggatttattacattctttctcagttcagttctagacagtctggcatccaccatagtcatgatttctctgttgcagaaattagtacctctgtcagagtatcagaagtatgtgtgtctgttgttaactttcactgtaatggtttgcttttgagttgaagtactaaatatgaccatattaatacaacaataatgattgactggggacaatcatgcatcattatacttctaaagcttcagatctctggtccttactggagaaacttgcgtggcattactcttgctttttaaattctcttacaaaatttgaatgcttatttgatttgtggtcttaataaaattttcctccagaaatgaattttgatcatttcaatttttcattttctgctagtacctataattgataggtgacttgaaatggtattagccttttcctttaagtttaaatctgcacttgtgatggctttctcgatgtctgttgagggaataaacattcaaccatgtatgtgaatcttctaagttttctgtttcattctccaactatttggaggtttgtctggctacatttcatcatgtcatcaagaagttgatttggattctatttgcaggatattgggaggtgttgttgtaatagcagcaaatgctggtggtgcatggagtcctattggtgctgttaccactactatgctgtggataaatggccaagtat

SEQ ID NO: 17 - DNA, 906 bp, Glycine max
agtattccttcaactccctggccttttgcaaacagatttcagcatccttccaatgagaaagactagcatataaatttgccagaccatgccaaatatcaaattcatttactttatcatactcaacctggaaagagttgagaaaatggaggtaagattgattttgcaatcagcaatttataattgaagtcccatgcatcaaccaaaaaaaaagaggaagcactaaaattcagtgtacaaaatgacaatgaaaaggaaatatcagaacatcataaaagaaatatataaaaaacaactttctttcttatacatttcttgactaatctttgaagtactcatttttaaagaatttaagataaagcttcccagacaacagctatctccataaaaacaacttacaggttgtctcacagttcttattaattggtattagatcttttcaccatgttccttagctgcaaacaagcaacatgggtggtgagatgtgatgtgccgatgttgttatgttcactcaacactatttaaaggactaatgcacagctagcctgcatgggtgccaaagctgttgtgcgtagtgagatgtaggatccaattacaaggtacaagactcgaggcccacattggaagtatgagattatgttgtgggggttcataaggccttggaatttctattcagaacagtagcttttgtagcattgttctctcaagattcttgttgagatcctacatcaactgtaaatatggtcaaagtaggcaatccttacctcatgagctaatttttaggattgagttatgctcaggccaaattcaagatagtatcagcgcttatcatagatacaatattttggccaccctcaactggctaaaaatctacctggccccacaattttcccagtgcaacatacttgg

SEQ ID NO: 18 - DNA, 989 bp, Glycine max
atcgccagcttgcatgcctgcagcattgatgcacactgaaggcaagaccaaatccttttgtttgtttgaagttttctatttaagcctgttgaacttaaccccaccctttttgatctaattgaggcggaggcaggaccgaggattgagtcccttgcctgcaaaactcctgtcctattgttgctgtgcctatgataggattatggatccaaattgggcccaataaggctttaatggatctgggagcatatgctcttatgtattctgtcttccttttcttatttttgtataatcttcctttttgttagataggagttgaatcctatcaattggtgcgttcattgagagaagtttaggggagattttggtgtatgcaaatgaacatgttttccctgtgactttttgggttcaattgaatccaaaatcctatcagcttatgcctgtttataggcctaaggctggatccacatgccgtcataaaagttttaagattgtaaagtcacatccatgaagtccatgaggtagagcataaaaccatcacaaacaggaatgaagcaatgagctaagatttatctgcttgtatagaaatatttgagtggacctactctaatataaacattttatattttcctgtgtgctatgccaggtgttttgtttgaaggacgtgggcaaaatgaagaagctctttgtgctactattaatgctatactactcgaaccaaactatgttccatgcaagatcttgatgggtgctttgtttcaaaaattgggtacaaagcatttggctattgcaagaagcttactgtctgatgcactccgaatagaacccacaaaccgcaaggcttggtataacttgggattgcttcacaaacatgagggccgaataagtgatgctgccgactgcttccaagcagcttccatgctcgaagaatctgatcccatcgaaagttttagctctttacctgacaggattcaattcctaaacagttaact

SEQ ID NO: 19 - DNA, 26 bp, Artificial Sequence
  Syntheic PCR primer
caccaaagtctaccaaggttaagaca

SEQ ID NO: 20 - DNA, 27 bp, Artificial Sequence
  Syntheic PCR primer
cagtatgttttccaactccttgaactt

SEQ ID NO: 21 - DNA, 28 bp, Artificial Sequence
  Syntheic PCR primer
acctttttcctctcctatttactattgc

SEQ ID NO: 22 - DNA, 28 bp, Artificial Sequence
  Syntheic PCR primer
actaattgtgccataaaattgtcatctt

SEQ ID NO: 23 - DNA, 25 bp, Artificial Sequence
  Syntheic PCR primer
gaaactcttctgggaccatgttaca

SEQ ID NO: 24 - DNA, 26 bp, Artificial Sequence
  Syntheic PCR primer
agcatcataattgttatctccattgg

SEQ ID NO: 25 - DNA, 24 bp, Artificial Sequence
  Syntheic PCR primer
aaggtctatttatgcaggcatgct

SEQ ID NO: 26 - DNA, 20 bp, Artificial Sequence
  Syntheic PCR primer
tgcctgttagcccacaagct

SEQ ID NO: 27 - DNA, 25 bp, Artificial Sequence
  Syntheic PCR primer
gttggatgtttggaagatgatctgt

SEQ ID NO: 28 - DNA, 26 bp, Artificial Sequence
  Syntheic PCR primer
ggtactatgatgtactgagcgtggat

SEQ ID NO: 29 - DNA, 23 bp, Artificial Sequence
  Syntheic PCR primer
ttgttccaatcctgacccaacta

SEQ ID NO: 30 - DNA, 24 bp, Artificial Sequence
  Syntheic PCR primer
tacaatctgagtcaaatgggcatt

SEQ ID NO: 31 - DNA, 26 bp, Artificial Sequence
  Syntheic PCR primer
gacctataggattagcagtccattcc

SEQ ID NO: 32 - DNA, 27 bp, Artificial Sequence
  Syntheic PCR primer
gagatttaggatatgacctttccacat

SEQ ID NO: 33 - DNA, 22 bp, Artificial Sequence
  Syntheic PCR primer
gagatgtctctggcggaaaaat

SEQ ID NO: 34 - DNA, 24 bp, Artificial Sequence
  Syntheic PCR primer
tgaaactctgctgctgtaaggaaa

SEQ ID NO: 35 - DNA, 27 bp, Artificial Sequence
  Syntheic PCR primer
agctggggaaagtcatttagaataatt

SEQ ID NO: 36 - DNA, 23 bp, Artificial Sequence
  Syntheic PCR primer
gggcatgctttttgtaaaaagaa

SEQ ID NO: 37 - DNA, 26 bp, Artificial Sequence
  Syntheic PCR primer
tgtctgctggttgaagtaacttatgg

SEQ ID NO: 38 - DNA, 26 bp, Artificial Sequence
  Syntheic PCR primer
gctgatagtttttgcatattcttcca

SEQ ID NO: 39 - DNA, 26 bp, Artificial Sequence
  Syntheic PCR primer
cttgcttacaaattcctccaactaaa

SEQ ID NO: 40 - DNA, 24 bp, Artificial Sequence
  Syntheic PCR primer
gcttaagaacaaccgagagctttt

SEQ ID NO: 41 - DNA, 25 bp, Artificial Sequence
  Syntheic PCR primer
tgtgtcatgtttgaaaagtgtgttg

SEQ ID NO: 42 - DNA, 22 bp, Artificial Sequence
  Syntheic PCR primer
tccatcctcgtcctctcaaaga

SEQ ID NO: 43 - DNA, 30 bp, Artificial Sequence
  Syntheic PCR primer
ttcaatgttgatagtgaagcattttactta

SEQ ID NO: 44 - DNA, 33 bp, Artificial Sequence
  Syntheic PCR primer
tcattttctatcttcttaacattgtttattcaa

SEQ ID NO: 45 - DNA, 34 bp, Artificial Sequence
  Syntheic PCR primer
cactgttgagagtattattctataaacaaaagaa

SEQ ID NO: 46 - DNA, 24 bp, Artificial Sequence
  Syntheic PCR primer
ctttgttgatttcaatgtcgcttt

SEQ ID NO: 47 - DNA, 28 bp, Artificial Sequence
  Syntheic PCR primer
catgaactgtgattacatattcttttgc

SEQ ID NO: 48 - DNA, 20 bp, Artificial Sequence
  Syntheic PCR primer
gctgccgaacatgatggtta

SEQ ID NO: 49 - DNA, 26 bp, Artificial Sequence
  Syntheic PCR primer
gattcaagagctccttcttacaaagg

SEQ ID NO: 50 - DNA, 28 bp, Artificial Sequence
  Syntheic PCR primer
tgcaaactacagaaaatttagaattcga

SEQ ID NO: 51 - DNA, 23 bp, Artificial Sequence
  Syntheic PCR primer
caccatgttccttagctgcaaac

SEQ ID NO: 52 - DNA, 22 bp, Artificial Sequence
  Syntheic PCR primer
acaacatcggcacatcacatct

SEQ ID NO: 53 - DNA, 25 bp, Artificial Sequence
  Syntheic PCR primer
ccaaactatgttccatgcaagatct

SEQ ID NO: 54 - DNA, 23 bp, Artificial Sequence
  Syntheic PCR primer
ttcttgcaatagccaaatgcttt

SEQ ID NO: 55 - DNA, 15 bp, Artificial Sequence
  Synthetic probe
acacagtagaaaatt

SEQ ID NO: 56 - DNA, 16 bp, Artificial Sequence
  Synthetic probe
acacagtagataattc

SEQ ID NO: 57 - DNA, 19 bp, Artificial Sequence
  Synthetic probe
ctagtcagttttgtttaat

SEQ ID NO: 58 - DNA, 15 bp, Artificial Sequence
  Synthetic probe
caactagtcaatttt

SEQ ID NO: 59 - DNA, 16 bp, Artificial Sequence
  Synthetic probe
aggcctgtttaggaaa

SEQ ID NO: 60 - DNA, 15 bp, Artificial Sequence
  Synthetic probe
aggcctgtttgggaa

SEQ ID NO: 61 - DNA, 14 bp, Artificial Sequence
  Synthetic probe
cctgctcacttaaa

SEQ ID NO: 62 - DNA, 13 bp, Artificial Sequence
  Synthetic probe
cctgcccacttaa

SEQ ID NO: 63 - DNA, 13 bp, Artificial Sequence
  Synthetic probe
aacggcaaattct

SEQ ID NO: 64 - DNA, 16 bp, Artificial Sequence
  Synthetic probe
aataacggtaaattct

SEQ ID NO: 65 - DNA, 14 bp, Artificial Sequence
  Synthetic probe
ctttggtctctcct

SEQ ID NO: 66 - DNA, 14 bp, Artificial Sequence
  Synthetic probe
ctttgatctctcct

SEQ ID NO: 67 - DNA, 16 bp, Artificial Sequence
  Synthetic probe
acacatcaagtccatg

SEQ ID NO: 68 - DNA, 16 bp, Artificial Sequence
  Synthetic probe
acacatcaggtccatg

SEQ ID NO: 69 - DNA, 15 bp, Artificial Sequence
  Synthetic probe
ccagactgtgttcaa

SEQ ID NO: 70 - DNA, 15 bp, Artificial Sequence
  Synthetic probe
ccccagattgtgttc

SEQ ID NO: 71 - DNA, 20 bp, Artificial Sequence
  Synthetic probe
ctagaaaatcgtataaatat

SEQ ID NO: 72 - DNA, 22 bp, Artificial Sequence
  Synthetic probe
atctagaaaatcatataaatat

SEQ ID NO: 73 - DNA, 15 bp, Artificial Sequence
  Synthetic probe
ttcgacatgggattt

SEQ ID NO: 74 - DNA, 16 bp, Artificial Sequence
  Synthetic probe
tcgacaagaatgggat

SEQ ID NO: 75 - DNA, 16 bp, Artificial Sequence
  Synthetic probe
ttattcgatgaaaatc

SEQ ID NO: 76 - DNA, 18 bp, Artificial Sequence
  Synthetic probe
ttattcaatgaaaatctt

SEQ ID NO: 77 - DNA, 16 bp, Artificial Sequence
  Synthetic probe
accacctgtccatatg

SEQ ID NO: 78 - DNA, 14 bp, Artificial Sequence
  Synthetic probe
acctgcccatatgt

SEQ ID NO: 79 - DNA, 17 bp, Artificial Sequence
  Synthetic probe
ccccatatgttatatct

SEQ ID NO: 80 - DNA, 16 bp, Artificial Sequence
  Synthetic probe
cccatatgttgtatct

SEQ ID NO: 81 - DNA, 18 bp, Artificial Sequence
  Synthetic probe
ctatacttatgaaatttc

SEQ ID NO: 82 - DNA, 17 bp, Artificial Sequence
  Synthetic probe
tatacttctgaaatttc

SEQ ID NO: 83 - DNA, 16 bp, Artificial Sequence
  Synthetic probe
ccggttgtatgtgatc

SEQ ID NO: 84 - DNA, 15 bp, Artificial Sequence
  Synthetic probe
ccggttgtatttgat

SEQ ID NO: 85 - DNA, 17 bp, Artificial Sequence
  Synthetic probe
acttttcaagagcaaat

SEQ ID NO: 86 - DNA, 17 bp, Artificial Sequence
  Synthetic probe
caaacttttgaagagca

SEQ ID NO: 87 - DNA, 16 bp, Artificial Sequence
  Synthetic probe
agcaacaagggtggtg

SEQ ID NO: 88 - DNA, 16 bp, Artificial Sequence
  Synthetic probe
aagcaacatgggtggt

SEQ ID NO: 89 - DNA, 17 bp, Artificial Sequence
  Synthetic probe
tttgaatcaaagcaccc

SEQ ID NO: 90 - DNA, 17 bp, Artificial Sequence
  Synthetic probe
atttttgaaacaaagca